SEQUENCE LISTING (236 sequences)

SEQ ID NO: 1 - DNA, 10 bp, Artificial Sequence
  Synthetic Sequence
aaccgcggct

SEQ ID NO: 2 - DNA, 10 bp, Artificial Sequence
  Synthetic Sequence
ggttataact

SEQ ID NO: 3 - DNA, 10 bp, Artificial Sequence
  Synthetic Sequence
ccaagtccgt

SEQ ID NO: 4 - DNA, 10 bp, Artificial Sequence
  Synthetic Sequence
ttggacttct

SEQ ID NO: 5 - DNA, 10 bp, Artificial Sequence
  Synthetic Sequence
cagtggatct

SEQ ID NO: 6 - DNA, 10 bp, Artificial Sequence
  Synthetic Sequence
tgacaagcgt

SEQ ID NO: 7 - DNA, 10 bp, Artificial Sequence
  Synthetic Sequence
cgagatatgt

SEQ ID NO: 8 - DNA, 10 bp, Artificial Sequence
  Synthetic Sequence
tagagcgcgt

SEQ ID NO: 9 - DNA, 10 bp, Artificial Sequence
  Synthetic Sequence
aacctgttgt

SEQ ID NO: 10 - DNA, 10 bp, Artificial Sequence
  Synthetic Sequence
ggttcaccgt

SEQ ID NO: 11 - DNA, 10 bp, Artificial Sequence
  Synthetic Sequence
cattgttgct

SEQ ID NO: 12 - DNA, 10 bp, Artificial Sequence
  Synthetic Sequence
tgccaccagt

SEQ ID NO: 13 - DNA, 12 bp, Artificial Sequence
  Synthetic Sequence
gccgcggttaga

SEQ ID NO: 14 - DNA, 12 bp, Artificial Sequence
  Synthetic Sequence
gttataaccaga

SEQ ID NO: 15 - DNA, 12 bp, Artificial Sequence
  Synthetic Sequence
cggacttggaga

SEQ ID NO: 16 - DNA, 12 bp, Artificial Sequence
  Synthetic Sequence
gaagtccaaaga

SEQ ID NO: 17 - DNA, 12 bp, Artificial Sequence
  Synthetic Sequence
gatccactgaga

SEQ ID NO: 18 - DNA, 12 bp, Artificial Sequence
  Synthetic Sequence
cgcttgtcaaga

SEQ ID NO: 19 - DNA, 12 bp, Artificial Sequence
  Synthetic Sequence
catatctcgaga

SEQ ID NO: 20 - DNA, 12 bp, Artificial Sequence
  Synthetic Sequence
cgcgctctaaga

SEQ ID NO: 21 - DNA, 12 bp, Artificial Sequence
  Synthetic Sequence
caacaggttaga

SEQ ID NO: 22 - DNA, 12 bp, Artificial Sequence
  Synthetic Sequence
cggtgaaccaga

SEQ ID NO: 23 - DNA, 12 bp, Artificial Sequence
  Synthetic Sequence
gcaacaatgaga

SEQ ID NO: 24 - DNA, 12 bp, Artificial Sequence
  Synthetic Sequence
ctggtggcaaga

SEQ ID NO: 25 - DNA, 10 bp, Artificial Sequence
  Synthetic Sequence
ctagcttgct

SEQ ID NO: 26 - DNA, 10 bp, Artificial Sequence
  Synthetic Sequence
tcgatccagt

SEQ ID NO: 27 - DNA, 10 bp, Artificial Sequence
  Synthetic Sequence
cctgaactgt

SEQ ID NO: 28 - DNA, 10 bp, Artificial Sequence
  Synthetic Sequence
ttcaggtcgt

SEQ ID NO: 29 - DNA, 10 bp, Artificial Sequence
  Synthetic Sequence
agtagagagt

SEQ ID NO: 30 - DNA, 10 bp, Artificial Sequence
  Synthetic Sequence
gacgagagct

SEQ ID NO: 31 - DNA, 10 bp, Artificial Sequence
  Synthetic Sequence
ctctgcctgt

SEQ ID NO: 32 - DNA, 10 bp, Artificial Sequence
  Synthetic Sequence
tctcattcgt

SEQ ID NO: 33 - DNA, 10 bp, Artificial Sequence
  Synthetic Sequence
acgccgcagt

SEQ ID NO: 34 - DNA, 10 bp, Artificial Sequence
  Synthetic Sequence
gtattatgct

SEQ ID NO: 35 - DNA, 10 bp, Artificial Sequence
  Synthetic Sequence
gatagatcgt

SEQ ID NO: 36 - DNA, 10 bp, Artificial Sequence
  Synthetic Sequence
agcgagctgt

SEQ ID NO: 37 - DNA, 12 bp, Artificial Sequence
  Synthetic Sequence
gcaagctagaga

SEQ ID NO: 38 - DNA, 12 bp, Artificial Sequence
  Synthetic Sequence
ctggatcgaaga

SEQ ID NO: 39 - DNA, 12 bp, Artificial Sequence
  Synthetic Sequence
cagttcaggaga

SEQ ID NO: 40 - DNA, 12 bp, Artificial Sequence
  Synthetic Sequence
cgacctgaaaga

SEQ ID NO: 41 - DNA, 12 bp, Artificial Sequence
  Synthetic Sequence
ctctctactaga

SEQ ID NO: 42 - DNA, 12 bp, Artificial Sequence
  Synthetic Sequence
gctctcgtcaga

SEQ ID NO: 43 - DNA, 12 bp, Artificial Sequence
  Synthetic Sequence
caggcagagaga

SEQ ID NO: 44 - DNA, 12 bp, Artificial Sequence
  Synthetic Sequence
cgaatgagaaga

SEQ ID NO: 45 - DNA, 12 bp, Artificial Sequence
  Synthetic Sequence
ctgcggcgtaga

SEQ ID NO: 46 - DNA, 12 bp, Artificial Sequence
  Synthetic Sequence
gcataatacaga

SEQ ID NO: 47 - DNA, 12 bp, Artificial Sequence
  Synthetic Sequence
cgatctatcaga

SEQ ID NO: 48 - DNA, 12 bp, Artificial Sequence
  Synthetic Sequence
cagctcgctaga

SEQ ID NO: 49 - DNA, 10 bp, Artificial Sequence
  Synthetic Sequence
agacttggct

SEQ ID NO: 50 - DNA, 10 bp, Artificial Sequence
  Synthetic Sequence
gagtccaagt

SEQ ID NO: 51 - DNA, 10 bp, Artificial Sequence
  Synthetic Sequence
cttaagccgt

SEQ ID NO: 52 - DNA, 10 bp, Artificial Sequence
  Synthetic Sequence
tccggattct

SEQ ID NO: 53 - DNA, 10 bp, Artificial Sequence
  Synthetic Sequence
ctgtattact

SEQ ID NO: 54 - DNA, 10 bp, Artificial Sequence
  Synthetic Sequence
tcacgccgct

SEQ ID NO: 55 - DNA, 10 bp, Artificial Sequence
  Synthetic Sequence
cagttccgct

SEQ ID NO: 56 - DNA, 10 bp, Artificial Sequence
  Synthetic Sequence
tgaccttagt

SEQ ID NO: 57 - DNA, 10 bp, Artificial Sequence
  Synthetic Sequence
ctaggcaagt

SEQ ID NO: 58 - DNA, 10 bp, Artificial Sequence
  Synthetic Sequence
tcgaatggct

SEQ ID NO: 59 - DNA, 10 bp, Artificial Sequence
  Synthetic Sequence
cttagtgtct

SEQ ID NO: 60 - DNA, 10 bp, Artificial Sequence
  Synthetic Sequence
tccgacacgt

SEQ ID NO: 61 - DNA, 12 bp, Artificial Sequence
  Synthetic Sequence
gccaagtctaga

SEQ ID NO: 62 - DNA, 12 bp, Artificial Sequence
  Synthetic Sequence
cttggactcaga

SEQ ID NO: 63 - DNA, 12 bp, Artificial Sequence
  Synthetic Sequence
cggcttaagaga

SEQ ID NO: 64 - DNA, 12 bp, Artificial Sequence
  Synthetic Sequence
gaatccggaaga

SEQ ID NO: 65 - DNA, 12 bp, Artificial Sequence
  Synthetic Sequence
gtaatacagaga

SEQ ID NO: 66 - DNA, 12 bp, Artificial Sequence
  Synthetic Sequence
gcggcgtgaaga

SEQ ID NO: 67 - DNA, 12 bp, Artificial Sequence
  Synthetic Sequence
gcggaactgaga

SEQ ID NO: 68 - DNA, 12 bp, Artificial Sequence
  Synthetic Sequence
ctaaggtcaaga

SEQ ID NO: 69 - DNA, 12 bp, Artificial Sequence
  Synthetic Sequence
cttgcctagaga

SEQ ID NO: 70 - DNA, 12 bp, Artificial Sequence
  Synthetic Sequence
gccattcgaaga

SEQ ID NO: 71 - DNA, 12 bp, Artificial Sequence
  Synthetic Sequence
gacactaagaga

SEQ ID NO: 72 - DNA, 12 bp, Artificial Sequence
  Synthetic Sequence
cgtgtcggaaga

SEQ ID NO: 73 - DNA, 10 bp, Artificial Sequence
  Synthetic Sequence
acttacatgt

SEQ ID NO: 74 - DNA, 10 bp, Artificial Sequence
  Synthetic Sequence
gtccgtgcgt

SEQ ID NO: 75 - DNA, 10 bp, Artificial Sequence
  Synthetic Sequence
aaggtaccgt

SEQ ID NO: 76 - DNA, 10 bp, Artificial Sequence
  Synthetic Sequence
ggaacgttct

SEQ ID NO: 77 - DNA, 10 bp, Artificial Sequence
  Synthetic Sequence
aattctgcgt

SEQ ID NO: 78 - DNA, 10 bp, Artificial Sequence
  Synthetic Sequence
ggcctcatgt

SEQ ID NO: 79 - DNA, 10 bp, Artificial Sequence
  Synthetic Sequence
aacaggaagt

SEQ ID NO: 80 - DNA, 10 bp, Artificial Sequence
  Synthetic Sequence
ggtgaaggct

SEQ ID NO: 81 - DNA, 10 bp, Artificial Sequence
  Synthetic Sequence
cctgtggcgt

SEQ ID NO: 82 - DNA, 10 bp, Artificial Sequence
  Synthetic Sequence
ttcacaatgt

SEQ ID NO: 83 - DNA, 10 bp, Artificial Sequence
  Synthetic Sequence
acacgagtgt

SEQ ID NO: 84 - DNA, 10 bp, Artificial Sequence
  Synthetic Sequence
gtgtagacgt

SEQ ID NO: 85 - DNA, 12 bp, Artificial Sequence
  Synthetic Sequence
catgtaagtaga

SEQ ID NO: 86 - DNA, 12 bp, Artificial Sequence
  Synthetic Sequence
cgcacggacaga

SEQ ID NO: 87 - DNA, 12 bp, Artificial Sequence
  Synthetic Sequence
cggtaccttaga

SEQ ID NO: 88 - DNA, 12 bp, Artificial Sequence
  Synthetic Sequence
gaacgttccaga

SEQ ID NO: 89 - DNA, 12 bp, Artificial Sequence
  Synthetic Sequence
cgcagaattaga

SEQ ID NO: 90 - DNA, 12 bp, Artificial Sequence
  Synthetic Sequence
catgaggccaga

SEQ ID NO: 91 - DNA, 12 bp, Artificial Sequence
  Synthetic Sequence
cttcctgttaga

SEQ ID NO: 92 - DNA, 12 bp, Artificial Sequence
  Synthetic Sequence
gccttcaccaga

SEQ ID NO: 93 - DNA, 12 bp, Artificial Sequence
  Synthetic Sequence
cgccacaggaga

SEQ ID NO: 94 - DNA, 12 bp, Artificial Sequence
  Synthetic Sequence
cattgtgaaaga

SEQ ID NO: 95 - DNA, 12 bp, Artificial Sequence
  Synthetic Sequence
cactcgtgtaga

SEQ ID NO: 96 - DNA, 12 bp, Artificial Sequence
  Synthetic Sequence
cgtctacacaga

SEQ ID NO: 97 - DNA, 11 bp, Artificial Sequence
  Synthetic Sequence
agcgctagcgt

SEQ ID NO: 98 - DNA, 11 bp, Artificial Sequence
  Synthetic Sequence
gatatcgatct

SEQ ID NO: 99 - DNA, 11 bp, Artificial Sequence
  Synthetic Sequence
cgcagacgcgt

SEQ ID NO: 100 - DNA, 11 bp, Artificial Sequence
  Synthetic Sequence
tatgagtatct

SEQ ID NO: 101 - DNA, 11 bp, Artificial Sequence
  Synthetic Sequence
aggtgcgtcgt

SEQ ID NO: 102 - DNA, 11 bp, Artificial Sequence
  Synthetic Sequence
gaacatacgct

SEQ ID NO: 103 - DNA, 11 bp, Artificial Sequence
  Synthetic Sequence
atcttagtcgt

SEQ ID NO: 104 - DNA, 11 bp, Artificial Sequence
  Synthetic Sequence
gctccgactgt

SEQ ID NO: 105 - DNA, 11 bp, Artificial Sequence
  Synthetic Sequence
ataccaagcgt

SEQ ID NO: 106 - DNA, 11 bp, Artificial Sequence
  Synthetic Sequence
gcgttggatct

SEQ ID NO: 107 - DNA, 11 bp, Artificial Sequence
  Synthetic Sequence
cttcacggcgt

SEQ ID NO: 108 - DNA, 11 bp, Artificial Sequence
  Synthetic Sequence
tcctgtaatct

SEQ ID NO: 109 - DNA, 13 bp, Artificial Sequence
  Synthetic Sequence
cgctagcgctaga

SEQ ID NO: 110 - DNA, 13 bp, Artificial Sequence
  Synthetic Sequence
gatcgatatcaga

SEQ ID NO: 111 - DNA, 13 bp, Artificial Sequence
  Synthetic Sequence
cgcgtctgcgaga

SEQ ID NO: 112 - DNA, 13 bp, Artificial Sequence
  Synthetic Sequence
gatactcataaga

SEQ ID NO: 113 - DNA, 13 bp, Artificial Sequence
  Synthetic Sequence
cgacgcacctaga

SEQ ID NO: 114 - DNA, 13 bp, Artificial Sequence
  Synthetic Sequence
gcgtatgttcaga

SEQ ID NO: 115 - DNA, 13 bp, Artificial Sequence
  Synthetic Sequence
cgactaagataga

SEQ ID NO: 116 - DNA, 13 bp, Artificial Sequence
  Synthetic Sequence
cagtcggagcaga

SEQ ID NO: 117 - DNA, 13 bp, Artificial Sequence
  Synthetic Sequence
cgcttggtataga

SEQ ID NO: 118 - DNA, 13 bp, Artificial Sequence
  Synthetic Sequence
gatccaacgcaga

SEQ ID NO: 119 - DNA, 13 bp, Artificial Sequence
  Synthetic Sequence
cgccgtgaagaga

SEQ ID NO: 120 - DNA, 13 bp, Artificial Sequence
  Synthetic Sequence
gattacaggaaga

SEQ ID NO: 121 - DNA, 11 bp, Artificial Sequence
  Synthetic Sequence
acatagcgcgt

SEQ ID NO: 122 - DNA, 11 bp, Artificial Sequence
  Synthetic Sequence
gtgcgatatct

SEQ ID NO: 123 - DNA, 11 bp, Artificial Sequence
  Synthetic Sequence
ccaacagatgt

SEQ ID NO: 124 - DNA, 11 bp, Artificial Sequence
  Synthetic Sequence
ttggtgagcgt

SEQ ID NO: 125 - DNA, 11 bp, Artificial Sequence
  Synthetic Sequence
cgcggttctct

SEQ ID NO: 126 - DNA, 11 bp, Artificial Sequence
  Synthetic Sequence
tataacctcgt

SEQ ID NO: 127 - DNA, 11 bp, Artificial Sequence
  Synthetic Sequence
agaatgcctgt

SEQ ID NO: 128 - DNA, 11 bp, Artificial Sequence
  Synthetic Sequence
gaggcattcgt

SEQ ID NO: 129 - DNA, 11 bp, Artificial Sequence
  Synthetic Sequence
cctcggtatct

SEQ ID NO: 130 - DNA, 11 bp, Artificial Sequence
  Synthetic Sequence
ttctaacgcgt

SEQ ID NO: 131 - DNA, 11 bp, Artificial Sequence
  Synthetic Sequence
atgaggctcgt

SEQ ID NO: 132 - DNA, 11 bp, Artificial Sequence
  Synthetic Sequence
gcagaatctgt

SEQ ID NO: 133 - DNA, 13 bp, Artificial Sequence
  Synthetic Sequence
cgcgctatgtaga

SEQ ID NO: 134 - DNA, 13 bp, Artificial Sequence
  Synthetic Sequence
gatatcgcacaga

SEQ ID NO: 135 - DNA, 13 bp, Artificial Sequence
  Synthetic Sequence
catctgttggaga

SEQ ID NO: 136 - DNA, 13 bp, Artificial Sequence
  Synthetic Sequence
cgctcaccaaaga

SEQ ID NO: 137 - DNA, 13 bp, Artificial Sequence
  Synthetic Sequence
gagaaccgcgaga

SEQ ID NO: 138 - DNA, 13 bp, Artificial Sequence
  Synthetic Sequence
cgaggttataaga

SEQ ID NO: 139 - DNA, 13 bp, Artificial Sequence
  Synthetic Sequence
caggcattctaga

SEQ ID NO: 140 - DNA, 13 bp, Artificial Sequence
  Synthetic Sequence
cgaatgcctcaga

SEQ ID NO: 141 - DNA, 13 bp, Artificial Sequence
  Synthetic Sequence
gataccgaggaga

SEQ ID NO: 142 - DNA, 13 bp, Artificial Sequence
  Synthetic Sequence
cgcgttagaaaga

SEQ ID NO: 143 - DNA, 13 bp, Artificial Sequence
  Synthetic Sequence
cgagcctcataga

SEQ ID NO: 144 - DNA, 13 bp, Artificial Sequence
  Synthetic Sequence
cagattctgcaga

SEQ ID NO: 145 - DNA, 11 bp, Artificial Sequence
  Synthetic Sequence
aaggatgatct

SEQ ID NO: 146 - DNA, 11 bp, Artificial Sequence
  Synthetic Sequence
ggaagcagcgt

SEQ ID NO: 147 - DNA, 11 bp, Artificial Sequence
  Synthetic Sequence
tcgtgaccgct

SEQ ID NO: 148 - DNA, 11 bp, Artificial Sequence
  Synthetic Sequence
ctacagttcgt

SEQ ID NO: 149 - DNA, 11 bp, Artificial Sequence
  Synthetic Sequence
atattcacgct

SEQ ID NO: 150 - DNA, 11 bp, Artificial Sequence
  Synthetic Sequence
gcgcctgtact

SEQ ID NO: 151 - DNA, 11 bp, Artificial Sequence
  Synthetic Sequence
cactacgatgt

SEQ ID NO: 152 - DNA, 11 bp, Artificial Sequence
  Synthetic Sequence
tgtcgtagcgt

SEQ ID NO: 153 - DNA, 11 bp, Artificial Sequence
  Synthetic Sequence
accacttatgt

SEQ ID NO: 154 - DNA, 11 bp, Artificial Sequence
  Synthetic Sequence
gttgtccgcgt

SEQ ID NO: 155 - DNA, 11 bp, Artificial Sequence
  Synthetic Sequence
atccatatcgt

SEQ ID NO: 156 - DNA, 11 bp, Artificial Sequence
  Synthetic Sequence
gcttgcgctct

SEQ ID NO: 157 - DNA, 13 bp, Artificial Sequence
  Synthetic Sequence
gatcatccttaga

SEQ ID NO: 158 - DNA, 13 bp, Artificial Sequence
  Synthetic Sequence
cgctgcttccaga

SEQ ID NO: 159 - DNA, 13 bp, Artificial Sequence
  Synthetic Sequence
gcggtcacgaaga

SEQ ID NO: 160 - DNA, 13 bp, Artificial Sequence
  Synthetic Sequence
cgaactgtagaga

SEQ ID NO: 161 - DNA, 13 bp, Artificial Sequence
  Synthetic Sequence
gcgtgaatataga

SEQ ID NO: 162 - DNA, 13 bp, Artificial Sequence
  Synthetic Sequence
gtacaggcgcaga

SEQ ID NO: 163 - DNA, 13 bp, Artificial Sequence
  Synthetic Sequence
catcgtagtgaga

SEQ ID NO: 164 - DNA, 13 bp, Artificial Sequence
  Synthetic Sequence
cgctacgacaaga

SEQ ID NO: 165 - DNA, 13 bp, Artificial Sequence
  Synthetic Sequence
cataagtggtaga

SEQ ID NO: 166 - DNA, 13 bp, Artificial Sequence
  Synthetic Sequence
cgcggacaacaga

SEQ ID NO: 167 - DNA, 13 bp, Artificial Sequence
  Synthetic Sequence
cgatatggataga

SEQ ID NO: 168 - DNA, 13 bp, Artificial Sequence
  Synthetic Sequence
gagcgcaagcaga

SEQ ID NO: 169 - DNA, 11 bp, Artificial Sequence
  Synthetic Sequence
actctatgcgt

SEQ ID NO: 170 - DNA, 11 bp, Artificial Sequence
  Synthetic Sequence
gtctcgcatct

SEQ ID NO: 171 - DNA, 11 bp, Artificial Sequence
  Synthetic Sequence
aagacgtctgt

SEQ ID NO: 172 - DNA, 11 bp, Artificial Sequence
  Synthetic Sequence
ggagtactcgt

SEQ ID NO: 173 - DNA, 11 bp, Artificial Sequence
  Synthetic Sequence
accggccatgt

SEQ ID NO: 174 - DNA, 11 bp, Artificial Sequence
  Synthetic Sequence
gttaattgcgt

SEQ ID NO: 175 - DNA, 11 bp, Artificial Sequence
  Synthetic Sequence
agtatcttcgt

SEQ ID NO: 176 - DNA, 11 bp, Artificial Sequence
  Synthetic Sequence
gacgctcctgt

SEQ ID NO: 177 - DNA, 11 bp, Artificial Sequence
  Synthetic Sequence
catgccatagt

SEQ ID NO: 178 - DNA, 11 bp, Artificial Sequence
  Synthetic Sequence
tgcattgcgct

SEQ ID NO: 179 - DNA, 11 bp, Artificial Sequence
  Synthetic Sequence
attggaacgct

SEQ ID NO: 180 - DNA, 11 bp, Artificial Sequence
  Synthetic Sequence
gccaaggtcgt

SEQ ID NO: 181 - DNA, 13 bp, Artificial Sequence
  Synthetic Sequence
cgcatagagtaga

SEQ ID NO: 182 - DNA, 13 bp, Artificial Sequence
  Synthetic Sequence
gatgcgagacaga

SEQ ID NO: 183 - DNA, 13 bp, Artificial Sequence
  Synthetic Sequence
cagacgtcttaga

SEQ ID NO: 184 - DNA, 13 bp, Artificial Sequence
  Synthetic Sequence
cgagtactccaga

SEQ ID NO: 185 - DNA, 13 bp, Artificial Sequence
  Synthetic Sequence
catggccggtaga

SEQ ID NO: 186 - DNA, 13 bp, Artificial Sequence
  Synthetic Sequence
cgcaattaacaga

SEQ ID NO: 187 - DNA, 13 bp, Artificial Sequence
  Synthetic Sequence
cgaagatactaga

SEQ ID NO: 188 - DNA, 13 bp, Artificial Sequence
  Synthetic Sequence
caggagcgtcaga

SEQ ID NO: 189 - DNA, 13 bp, Artificial Sequence
  Synthetic Sequence
ctatggcatgaga

SEQ ID NO: 190 - DNA, 13 bp, Artificial Sequence
  Synthetic Sequence
gcgcaatgcaaga

SEQ ID NO: 191 - DNA, 13 bp, Artificial Sequence
  Synthetic Sequence
gcgttccaataga

SEQ ID NO: 192 - DNA, 13 bp, Artificial Sequence
  Synthetic Sequence
cgaccttggcaga

SEQ ID NO: 193 - DNA, 34 bp, Artificial Sequence
  Synthetic Sequence
tacactctttccctacacgacgctcttccgatct

SEQ ID NO: 194 - DNA, 33 bp, Artificial Sequence
  Synthetic Sequence
cactgacctcaagtctgcacacgagaaggctag

SEQ ID NO: 195 - DNA, 32 bp, Artificial Sequence
  Synthetic Sequence
cactgacctcaagtctgcacacgagaaggcta

SEQ ID NO: 196 - DNA, 31 bp, Artificial Sequence
  Synthetic Sequence
cactgacctcaagtctgcacacgagaaggct

SEQ ID NO: 197 - DNA, 30 bp, Artificial Sequence
  Synthetic Sequence
cactgacctcaagtctgcacacgagaaggc

SEQ ID NO: 198 - DNA, 29 bp, Artificial Sequence
  Synthetic Sequence
cactgacctcaagtctgcacacgagaagg

SEQ ID NO: 199 - DNA, 13 bp, Artificial Sequence
  Synthetic Sequence
atcgagtgcacat

SEQ ID NO: 200 - DNA, 13 bp, Artificial Sequence
  Synthetic Sequence
atagctcacgtgt

SEQ ID NO: 201 - DNA, 14 bp, Artificial Sequence
  Synthetic Sequence
gatagctcacgtgt

SEQ ID NO: 202 - DNA, 15 bp, Artificial Sequence
  Synthetic Sequence
agatagctcacgtgt

SEQ ID NO: 203 - DNA, 16 bp, Artificial Sequence
  Synthetic Sequence
tagatagctcacgtgt

SEQ ID NO: 204 - DNA, 17 bp, Artificial Sequence
  Synthetic Sequence
ctagatagctcacgtgt

SEQ ID NO: 205 - DNA, 23 bp, Artificial Sequence
  Synthetic Sequence
cgagggagcttccagggggaaac

SEQ ID NO: 206 - DNA, 22 bp, Artificial Sequence
  Synthetic Sequence
ttgggcgctcttccgcttcctc

SEQ ID NO: 207 - DNA, 33 bp, Artificial Sequence
  Synthetic Sequence
tacactctttccctacacgacgctcttccgatc

SEQ ID NO: 208 - DNA, 34 bp, Artificial Sequence
  Synthetic Sequence
cactgacctcaagtctgcacacgagaaggctaga

SEQ ID NO: 209 - DNA, 14 bp, Artificial Sequence
  Synthetic Sequence
tatcgagtgcacat

SEQ ID NO: 210 - DNA, 12 bp, Artificial Sequence
  Synthetic Sequence
tagctcacgtgt

SEQ ID NO: 211 - DNA, 33 bp, Artificial Sequence; identical to SEQ ID NO: 207
  Synthetic Sequence

SEQ ID NO: 212 - DNA, 32 bp, Artificial Sequence; identical to SEQ ID NO: 195
  Synthetic Sequence

SEQ ID NO: 213 - DNA, 14 bp, Artificial Sequence
  Synthetic Sequence
gatagctcacgtgt

SEQ ID NO: 214 - DNA, 32 bp, Artificial Sequence
  Synthetic Sequence
tacactctttccctacacgacgctcttccgat

SEQ ID NO: 215 - DNA, 33 bp, Artificial Sequence; identical to SEQ ID NO: 194
  Synthetic Sequence

SEQ ID NO: 216 - DNA, 15 bp, Artificial Sequence
  Synthetic Sequence
ctatcgagtgcacat

SEQ ID NO: 217 - DNA, 13 bp, Artificial Sequence
  Synthetic Sequence
atagctcacgtgt

SEQ ID NO: 218 - DNA, 31 bp, Artificial Sequence
  Synthetic Sequence
tacactctttccctacacgacgctcttccga

SEQ ID NO: 219 - DNA, 32 bp, Artificial Sequence; identical to SEQ ID NO: 195
  Synthetic Sequence

SEQ ID NO: 220 - DNA, 16 bp, Artificial Sequence
  Synthetic Sequence
tctatcgagtgcacat

SEQ ID NO: 221 - DNA, 14 bp, Artificial Sequence
  Synthetic Sequence
gatagctcacgtgt

SEQ ID NO: 222 - DNA, 31 bp, Artificial Sequence; identical to SEQ ID NO: 218
  Synthetic Sequence

SEQ ID NO: 223 - DNA, 30 bp, Artificial Sequence; identical to SEQ ID NO: 197
  Synthetic Sequence

SEQ ID NO: 224 - DNA, 16 bp, Artificial Sequence
  Synthetic Sequence
tctatcgagtgcacat

SEQ ID NO: 225 - DNA, 16 bp, Artificial Sequence
  Synthetic Sequence
tagatagctcacgtgt

SEQ ID NO: 226 - DNA, 99 bp, Artificial Sequence
  Synthetic Sequence
gtggagaagctcccaaccaagctctcttgaggatcttgaaggaaactgaattcaaaaagatcaaagtgctgggctccggtgcgttcggcacggtgtata

SEQ ID NO: 227 - DNA, 99 bp, Artificial Sequence
  Synthetic Sequence
acattgcatacattcgaaagaccctagccttagataaaactgagcaagaggctttggagtatttcatgaaacaaatgaatgatgcacatcatggtggct

SEQ ID NO: 228 - DNA, 99 bp, Artificial Sequence
  Synthetic Sequence
acaagatttacctctattgttggatcatattcgtccacaaaatgattctgaattagctgtatcgtcaaggcactcttgcctacgccaccagctccaact

SEQ ID NO: 229 - DNA, 99 bp, Artificial Sequence
  Synthetic Sequence
tgtattggtctctcatggcactgtactcttcttgtccagctgtatccagtatgtccaacaaacaggtttcaccatctataaccacttgttttctgtaag

SEQ ID NO: 230 - DNA, 99 bp, Artificial Sequence
  Synthetic Sequence
gacccactccatcgagatttcactgtagctagaccaaaatcacctatttttactgtgaggtcttcatgaagaaatatatctgaggtgtagtaagtaaag

SEQ ID NO: 231 - DNA, 99 bp, Artificial Sequence
  Synthetic Sequence
tccttactcatggtcggatcacaaagatttgtgattttggtctagccagagacatcaagaatgattctaattatgtggttaaaggaaacgtgagtaccc

SEQ ID NO: 232 - DNA, 99 bp, Artificial Sequence
  Synthetic Sequence
gagacaatgaattaagggaaaatgacaaagaacagctcaaagcaatttctacacgagatcctctctctgaaatcactgagcaggagaaagattttctat

SEQ ID NO: 233 - DNA, 99 bp, Artificial Sequence; identical to SEQ ID NO: 228
  Synthetic Sequence

SEQ ID NO: 234 - DNA, 99 bp, Artificial Sequence
  Synthetic Sequence
ttttgggctggccaaactgctgggtgcggaagagaaagaataccatgcagaaggaggcaaagtaaggaggtggctttaggtcagccagcattttcctga

SEQ ID NO: 235 - DNA, 99 bp, Artificial Sequence
  Synthetic Sequence
cccagaaggtgagaaagttaaaattcccgtcgctatcaaggaattaagagaagcaacatctccgaaagccaacaaggaaatcctcgatgtgagtttctg

SEQ ID NO: 236 - DNA, 99 bp, Artificial Sequence
  Synthetic Sequence
ctccaggaagcctacgtgatggccagcgtggacaacccccacgtgtgccgcctgctgggcatctgcctcacctccaccgtgcagctcatcacgcagctc

(19) United States
(12) Patent Application Publication
(10) Pub. No.: US 2023/0407370 A1
(43) Pub. Date: Dec. 21, 2023

(54) GEOMETRIC SYNTHESIS METHODS AND COMPOSITIONS FOR DOUBLE-STRANDED NUCLEIC ACID SEQUENCING

(71) Applicant: CAMENA BIOSCIENCE LIMITED, Essex (GB)

(72) Inventor: Neil BELL, Essex (GB)

(73) Assignee: CAMENA BIOSCIENCE LIMITED, Essex (GB)

(21) Appl. No.: 18/253,864

(22) PCT Filed: Nov. 22, 2021
(86) PCT No.: PCT/US2021/060328

§ 371 Date: May 22, 2023

Related U.S. Application Data
(60) Provisional application No. 63/116,552, filed on Nov. 20, 2020.

(51) Int. Cl. C12Q 1/6806; C12Q 1/6869

(52) U.S. Cl. CPC C12Q 1/6806; C12Q 1/6869

(57) ABSTRACT

The present disclosure provides compositions, kits and methods for sequencing double-stranded nucleic acids. The compositions, kits and methods comprise partially double-stranded identifier molecules and partially double-stranded adapter molecules. The compositions, kits and methods can be used to determine the abundance and/or identity of specific transcripts in a plurality of double-stranded nucleic acids, as well as identifying the frequency of mutations within certain transcripts in the plurality of double-stranded nucleic acids.

### CROSS-REFERENCE TO RELATED APPLICATIONS

This application claims priority to, and the benefit of, U.S. Provisional Application No. 63/116,552, filed Nov. 20, 2020, the contents of which are incorporated herein by reference in their entireties for all purposes.

### SEQUENCE LISTING

The instant application contains a Sequence Listing which has been submitted in ASCII format via EFS-Web and is hereby incorporated by reference in its entirety. Said ASCII copy, created on Nov. 21, 2021, is named “DNWR-008_SeqList.txt” and is about 46,527 bytes in size.

### BACKGROUND

There is a need in the art for sensitive and specific methods and materials to detect mutations (variations) in circulating tumor DNA or for the identification of variants in complex mixed metagenomic samples as well as many other applications. Next generation sequencing (NGS) platforms provide enormous depth of sequence data across many genes simultaneously.

Often, however, due to amplification artifact combined with intrinsic errors, the fidelity of variant calling is compromised. The present disclosure provides methods, compositions and kits for the sensitive and specific identification of variant alleles in complex DNA samples. The present disclosure provides methods, compositions and kits that can be used to identify variants at the sensitivity appropriate to the expected degree of variation by controlling the number of potential bar codes and the depth of sequence read coverage.

### BACKGROUND

The present disclosure provides pluralities of partially double-stranded identifier molecules, wherein the partially double-stranded identifier molecules comprise: a double-stranded region comprising an identifier sequence; and a first overhang; wherein the plurality comprises at least about 12 species of the partially double-stranded adapter molecules, wherein each species of partially double-stranded adapter molecules comprises an identifier sequence that is different from the identifier sequence of any other species of partially double-stranded adapter molecules in the plurality, wherein the identifier sequence of one species of partially double-stranded identifier molecules will have a hamming distance of at least about two to any other identifier sequence of any other species of partially double-stranded identifier molecules in the plurality.

In some aspects, the partially double-stranded identifier molecules further comprise a second overhang.

In some aspects the first and second overhangs are a) 5′ overhangs; or b) 3′ overhangs.

In some aspects, the identifier sequence spans the entire double-stranded region. In some aspects, the identifier sequence spans a portion of the double-stranded region.

In some aspects, the identifier sequence is: a) about 9 nucleotides in length; b) about 10 nucleotides in length: c) about 11 nucleotides in length; d) about 12 nucleotides in length; e) about 19 nucleotides in length; f) about 20 nucleotides in length; g) about 21 nucleotides in length; or h) about 22 nucleotides in length.

In some aspects, the first overhang and/or the second overhang is about 1 nucleotide in length. In some aspects, the first overhang and/or the second overhang is about 1 nucleotide in length, and the first overhang and/or the second overhang is: a) an adenine or a thymine; or b) a guanosine or a cytosine.

In some aspects, the first overhang and/or the second overhang is: a) about 2 nucleotides in length; b) about 3 nucleotides in length; c) about 4 nucleotides in length; or d) about 5 nucleotides in length.

In some aspects, the partially double-stranded identifier molecules comprise DNA.

In some aspects, a plurality comprises: a) at least about 24 species of the partially double-stranded identifier molecules; b) at least about 48 species of the partially double-stranded identifier molecules; or c) at least about 96 species of the partially double-stranded identifier molecules.

The present disclosure provides pluralities of partially double-stranded adapter molecules, wherein the partially double-stranded adapter molecules comprise: a double-stranded region; an overhang; a single-stranded 5′ arm; and a single-stranded 3′ arm; wherein the single-stranded 5′ arm comprises at least one amplification primer binding site and the single-stranded 3′ arm comprises at least one amplification primer binding site. In some aspects, the double-stranded region comprises an identifier sequence. In some aspects, a plurality comprises at least about 12 species of the partially double-stranded adapter molecules, wherein each species of partially double-stranded adapter molecules comprises an identifier sequence that is different from the identifier sequence of any other species of partially double-stranded adapter molecules in the plurality.

In some aspects, the overhang is: a) a 5′ overhang; or b) a 3′ overhang.

In some aspects, an overhang is about 1 nucleotide in length. In some aspects, an overhang is about 1 nucleotide in length, and wherein the overhang is: a) an adenine or a thymine; or b) a guanosine or cytosine.

In some aspects, an overhang is: a) about 2 nucleotides in length; b) about 3 nucleotides in length; c) about 4 nucleotides in length; d) about 5 nucleotides in length.

In some aspects, an identifier sequence is: a) about 9 nucleotides in length; b) about 10 nucleotides in length: c) about 11 nucleotides in length; d) about 12 nucleotides in length; e) about 19 nucleotides in length; f) about 20 nucleotides in length; g) about 21 nucleotides in length; or h) about 22 nucleotides in length.

In some aspects, partially double-stranded adapter molecules comprise DNA.

The present disclosure provides methods of sequencing a plurality of double-stranded target nucleic acids comprising: a) contacting the plurality of double-stranded target nucleic acids with the plurality of partially double-stranded identifier molecules of any one of claims 1-9 and at least one ligase such that a partially double-stranded identifier molecule is ligated to each end of the double-stranded target nucleic acids in the plurality of double-stranded target nucleic acids; b) contacting the products of step (a) with the plurality of partially double-stranded adapter molecules of any one of claims 10-16 and at least one ligase such that a partially double-stranded adapter molecule is ligated to each end of the products of step (a); and c) sequencing the products of step (b).

In some aspects, the ligation products in step (a) comprise: a) at least 10% of the combinations of two species of partially double-stranded identifier molecules; b) at least 20% of the combinations of two species of partially double-stranded identifier molecules; c) at least 30% of the combinations of two species of partially double-stranded identifier molecules; d) at least 40% of the combinations of two species of partially double-stranded identifier molecules; e) at least 50% of the combinations of two species of partially double-stranded identifier molecules; f) at least 60% of the combinations of two species of partially double-stranded identifier molecules; g) at least 70% of the combinations of two species of partially double-stranded identifier molecules; h) at least 80% of the combinations of two species of partially double-stranded identifier molecules; i) at least 90% of the combinations of two species of partially double-stranded identifier molecules; or j) each of the combinations of two species of partially double-stranded identifier molecules.

In some aspects, the methods further comprise after step (b) and prior to step (c), constructing a sequencing library using the products of step (b).

In some aspects, step (a) and step (b) are performed sequentially or are performed concurrently.

In some aspects, the methods further comprise after step (b) and prior to step (c), amplifying the products of step (b). In some aspects, amplifying the products of step (b) comprises contacting the products of step (b) with amplification primers that bind to amplification primer binding sites in the partially double-stranded adapter molecules and at least one polymerase.

In some aspects, the methods further comprise determining the abundance and/or the identity of specific transcripts in the plurality of double-stranded target nucleic acids using the sequencing data generated in step (c). In some aspects, determining the abundance and/or the identity of specific transcripts in the plurality of double-stranded target nucleic acids using the sequencing data generated in step (c) comprises correcting for errors using the identifier sequences of the ligated partially double-stranded identifier molecules. In some aspects, the errors comprise amplification errors, sample preparation errors, sequencing errors or any combination thereof; In some aspects, determining the abundance and/or the identity of specific transcripts in the plurality of double-stranded target nucleic acids using the sequencing data generated in step (c) comprises creating consensus sequences using identifier sequences of the ligated partially double-stranded identifier molecules.

In some aspects, determining the abundance and/or identity of specific transcripts in the plurality of double-stranded target nucleic acids using the sequencing data generated in step (c) comprises grouping the sequencing reads obtained in step (c) by the ligated identifier sequences in the sequencing reads, grouping the sequencing reads obtained in step (c) by the specific genomic sequence that the sequencing reads most likely correspond to, or any combination thereof.

In some aspects, determining the abundance and/or identify of specific transcripts in the plurality of double-stranded target nucleic acids can comprise determining the frequency of one or more mutations in a specific transcript in the plurality of double-stranded target nucleic acid, In some aspects, the one or more mutations comprise one or more insertions, one or more deletion-insertions, one or more duplications, one or more inversions, one or more repeat expansions or any combination thereof.

The present disclosure provides kits comprising at least one plurality of partially double-stranded identifier molecules of the present disclosure. In some aspects, the kits can further comprise at least one plurality of partially double-stranded adapter molecules of the present disclosure.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a schematic overview of the methods and compositions of the present disclosure.

FIG. 2 is a schematic overview of the methods and compositions of the present disclosure.

FIG. 3 is a schematic overview of partially double-stranded adapter molecules of the present disclosure.

FIG. 4 is a schematic of an exemplary sequencing data analysis workflow of the present disclosure.

FIG. 5 shows the results of an experiment using the methods and compositions of the present disclosure, specifically identifier molecules and adapter molecules comprising multiple base overhangs. The nucleic acid sequences shown in this figure correspond to SEQ ID NOs: 197-208.

FIG. 6 shows the results of an experiment using the methods and compositions of the present disclosure, specifically identifier molecules and adapter molecules comprising single base overhangs with varying sizes of double-stranded regions. The nucleic acid sequences shown in this figure correspond to SEQ ID NOs: 211-229.

FIG. 7 is a schematic comparison between existing next generation sequencing barcode compositions and methods that rely on the use of pre-pooled, degenerate barcodes and the compositions and the methods of the present disclosure.

FIG. 8 shows heatmaps generated for the coverage of each UMI created using the sequencing compositions and methods prior (left) and post (middle) error correction; the difference for the coverage between the UMIs prior and post error-correction is also shown (right), showing regions were UMI coverage decreased and increased. CorrectUmis (fgbio tools) was used for the UMI error-correction.

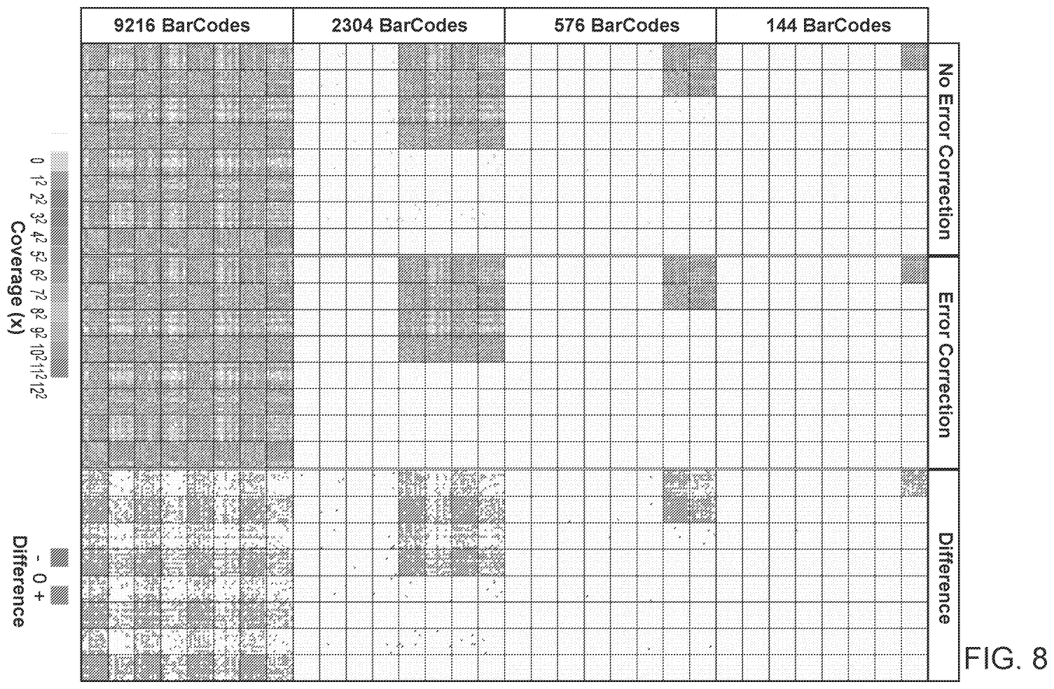

FIG. 9 shows an example Bioanalyzer trace from sequencing libraries assembled using amplicons prepare from gDNA (Quantitative Multiplex Reference Standard, Horizon Discovery) using AmpliSeq primers and partially double stranded identifier molecules and adapter molecules of the present disclosure.

FIG. 10 shows the sequencing results for the EGFR4 gene and the measured mutant frequencies for a DNA base change of GGC→AGC obtained using existing NGS methods and the sequencing methods of the present disclosure. The nucleic acid sequence shown in this figure corresponds to SEQ ID NO: 226.

FIG. 11 shows the sequencing results for the PI3KCA10 gene and the measured mutant frequencies for a DNA base change of CAT→CGT obtained using existing NGS methods and the sequencing methods of the present disclosure. The nucleic acid sequence shown in this figure corresponds to SEQ ID NO: 227.

FIG. 12 shows the sequencing results for the KRAS1 gene and the measured mutant frequencies for a DNA base change of GGC→GAC obtained using existing NGS methods and the sequencing methods of the present disclosure. The nucleic acid sequence shown in this figure corresponds to SEQ ID NO: 228.

FIG. 13 shows the sequencing results for the NRAS gene and the measured mutant frequencies for a DNA base change of CAA→AAA obtained using existing NGS methods and the sequencing methods of the present disclosure. The nucleic acid sequence shown in this figure corresponds to SEQ ID NO: 229.

FIG. 14 shows the sequencing results for the BRAF gene and the measured mutant frequencies for a DNA base change of CTG→CAG obtained using existing NGS methods and the sequencing methods of the present disclosure. The nucleic acid sequence shown in this figure corresponds to SEQ ID NO: 230.

FIG. 15 shows the sequencing results for the KIT gene and the measured mutant frequencies for a DNA base change of GAC→GTC obtained using existing NGS methods and the sequencing methods of the present disclosure. The nucleic acid sequence shown in this figure corresponds to SEQ ID NO: 231.

FIG. 16 shows the sequencing results for the PI3KCA7 gene and the measured mutant frequencies for a DNA base change of GAG→AAG obtained using existing NGS methods and the sequencing methods of the present disclosure. The nucleic acid sequence shown in this figure corresponds to SEQ ID NO: 232.

FIG. 17 shows the sequencing results for the KRAS1 gene and the measured mutant frequencies for a DNA base change of GGT→GAT obtained using existing NGS methods and the sequencing methods of the present disclosure. The nucleic acid sequence shown in this figure corresponds to SEQ ID NO: 233.

FIG. 18 shows the sequencing results for the EGFR8 gene and the measured mutant frequencies for a DNA base change of CTG→CGG obtained using existing NGS methods and the sequencing methods of the present disclosure. The nucleic acid sequence shown in this figure corresponds to SEQ ID NO: 234.

FIG. 19 shows the sequencing results for the EGFR5 gene and the measured mutant frequencies for a DNA base change of AAGGAATTAAGAGAAGCA→AA obtained using existing NGS methods and the sequencing methods of the present disclosure. The nucleic acid sequence shown in this figure corresponds to SEQ ID NO: 235.

FIG. 20 shows the sequencing results for the EGFR6 gene and the measured mutant frequencies for a DNA base change of ACG→ATG obtained using existing NGS methods and the sequencing methods of the present disclosure. The nucleic acid sequence shown in this figure corresponds to SEQ ID NO: 236.

### DETAILED DESCRIPTION

Existing methods for generating Next Generation Sequencing (NGS) libraries provide unique molecular identifiers (UMI) to individual fragments of DNA. When the DNA is amplified, the sequence of the original molecule may be unambiguously identified, because each of the multiple copies will have the same UMI as the original DNA fragment. Not only does this allow normalization after amplification for quantitative use, but also reduces sequencing error by providing a consensus.

The present disclosure provides improved double-stranded nucleic acid sequencing methods by adding an adjustable number of available barcodes. In this approach, modular adapter and identifier molecules are simultaneously ligated to complex mixtures of individual target DNA fragments to generate an NGS library. Individual identifier molecules are added to DNA fragments though single base overhangs (e.g. A/T). Simultaneously, partially double-stranded Y-shaped adapter molecules are ligated to the ends of identifier molecules already attached to the target DNA molecules using an overhanging sequence, which is reverse complementary on the identifier and adapter molecules.

In some aspects, the identifier molecules are a small subset of all possible 11- or 20-mer base pair identifier sequences and are selected to be unambiguous when sequenced. In a non-limiting example, an identifier set may comprise only 12 individual sequences, but because they will be randomly affixed to either end of a target DNA molecule, there will be 144 barcode possibilities (12×12=144). Without wishing to be bound by theory, because of the randomness of the target DNA fragment length and genomic location, and the unique encoding of each strand from the duplex, the resulting barcodes allow the unique identification of the original DNA molecules. For rare events, such as mutated bases in a circulating free DNA that may be associated with cancer, the number of barcodes employed can be adjusted, along with the depth of sequencing, to provide the appropriate sensitivity for the specific application. Without wishing to be bound by theory, higher sensitivity in deep sequencing will require a larger number of possible barcodes. For example, one may want to use a set of identifier molecules that is has 96 different identifier sequences allowing for a total of 9216 (96×96=9216) distinct barcodes.

In some aspects, individual libraries are uniquely identified from a mix of libraries by an index identifier that is added during amplification carried by amplification primers. Additionally, platform specific adapter molecules are incorporated, allowing the user to employ any existing NGS systems, including, but not limited to, Illumina, Oxford Nanopore or Pacific Bioscience.

Partially Double-Stranded Identifier Molecules

The present disclosure provides partially double-stranded identifier molecules. Partially double-stranded identifier molecules are nucleic acid molecules comprising at least one double-stranded region and at least one single stranded region. In some aspects, a partially double-stranded identifier molecule is a nucleic acid molecule comprising one double-stranded region and one single-stranded region. In some aspects, a partially double-stranded identifier molecule is a nucleic acid molecule comprising one doubles-stranded region and two single-stranded regions.

In some aspects a partially double-stranded identifier molecule comprises DNA. In some aspects, a partially double-stranded identifier molecule comprises RNA. In some aspects, a partially double-stranded identifier molecule can comprise XNA. In some aspects, a partially double-stranded identifier molecule comprises any combination of DNA, RNA and XNA.

As used herein, the term “XNA” is used to refer to xeno nucleic acids. As would be appreciated by the skilled artisan, xeno nucleic acids are synthetic nucleic acid analogues comprising a different sugar backbone than the natural nucleic acids DNA and RNA. XNAs can include, but are not limited to, 1,5-anhydrohexitol nucleic acid (HNA), Cyclohexene nucleic acid (CeNA), Threose nucleic acid (TNA), Glycol nucleic acid (GNA), Locked nucleic acid (LNA), Peptide nucleic acid (PNA) and FANA (Fluoro Arabino nucleic acid).

In some aspects, a partially double-stranded identifier molecule can comprise an identifier sequence, also referred to herein as an identifier nucleic acid sequence, a barcode sequence or a hemi-barcode sequence. As would be appreciated by the skilled artisan, an identifier sequence is a nucleic acid sequence that can be used as part of a sequencing method to identify individual molecules within a sample. An identifier sequence can comprise a degenerate, a semi-degenerate or discrete (non-degenerate) nucleic acid sequence.

In some aspects, an identifier sequence can be a nucleic acid sequence that is known not to occur or that occurs infrequently in the genome of an organism from which a sample is derived. In a non-limiting example, an identifier sequence can be a nucleic acid sequence that is known not to occur or that occurs infrequently in the human genome.

In some aspects, a partially double-stranded identifier molecule can comprise one overhang. The overhang can be a 3′ overhang or a 5′ overhang. In some aspects, a partially double-stranded identifier molecule can comprise two overhangs. The overhangs can be 3′ overhangs or 5′ overhangs.

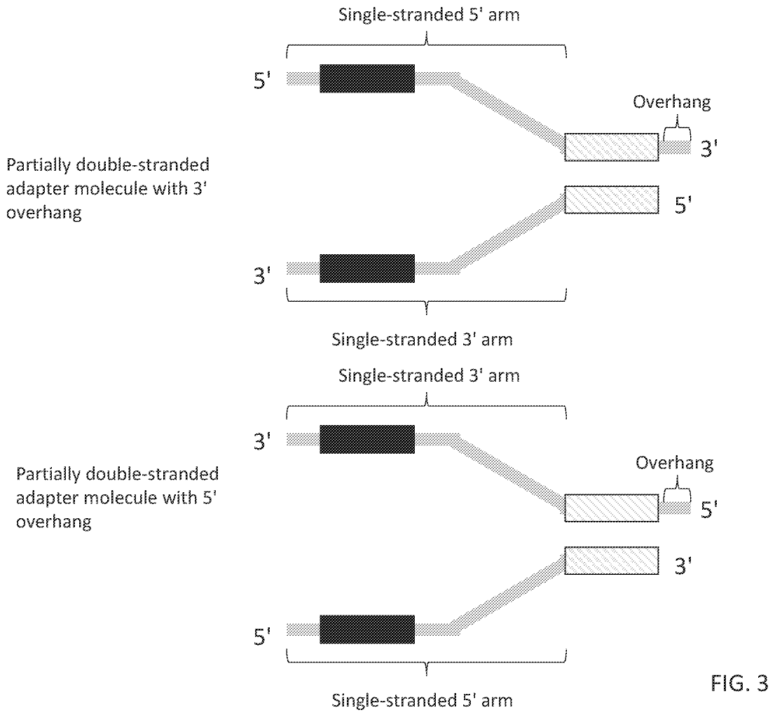

As would be appreciated by the skilled artisan, an “overhang”, in the context of a partially double-stranded nucleic acid molecule refers to a single-stranded region of a partially-double stranded nucleic acid molecule located at a terminus of the partially double-stranded nucleic acid molecule for which there is no single-stranded region located on the opposite strand. FIG. 3 shows 3′ and 5′ overhangs in exemplary partially double-stranded nucleic acid molecules, namely partially double-stranded adapter molecules of the present disclosure, which are described in further detailed herein.

As used herein, the term 5′ overhang is used to refer to a single-stranded region of a partially double-stranded nucleic acid molecule that is located at the 5′ terminus of one of the strands.

As used herein, the term 3′ overhang is used to refer to a single-stranded region of a partially double-stranded nucleic acid molecule that is located at the 3′ terminus of one of the strands.

In some aspects, a partially double-stranded identifier molecule can comprise an identifier sequence and one overhang. In some aspects, the overhang can be a 3′ overhang. In some aspects, the overhang can be a 5′ overhang.

In some aspects, a partially double-stranded identifier molecule can comprise an identifier sequence and two overhangs. In some aspects, the overhangs can be 3′ overhangs. In some aspects, the overhang can be a 5′ overhangs.

In some aspects of the compositions of the present disclosure a 5′ overhang of a partially double-stranded identifier molecule can be about 1 nucleotide in length, or about 2 nucleotides in length, or about 3 nucleotides in length, or about 4 nucleotides in length, or about 5 nucleotides in length. In some aspects of the compositions of the present disclosure a 5′ overhang can be at least about 1 nucleotide, or at least about 2 nucleotides, or at least about 3 nucleotides, or at least about 4 nucleotides, or at least about 5 nucleotides, or at least about 6 nucleotides, or at least about 7 nucleotides, or at least about 8 nucleotides, or at least about 9 nucleotides, or at least about 10 nucleotides in length.

In some aspects, a 5′ overhang of a partially double-stranded identifier molecule is no more than 1, or no more than 2, or no more than 3, or no more than 4, or no more than 5, or no more than 6, or no more than 7, or no more than 8, or no more than 9, or no more than 10 nucleotides, or no more than 11 nucleotides, or no more than 12 nucleotides, or no more than 13 nucleotide, or no more than 14 nucleotides, or no more than 15 nucleotides, or no more than 16 nucleotides, or no more than 17 nucleotides, or no more than 18 nucleotides, or no more than 19 nucleotides, or no more than 20 nucleotides in length.

In some aspects of the compositions of the present disclosure a 3′ overhang of a partially double-stranded identifier molecule can be about 1 nucleotide in length, or about 2 nucleotides in length, or about 3 nucleotides in length, or about 4 nucleotides in length, or about 5 nucleotides in length. In some aspects of the compositions of the present disclosure a 3′ overhang can be at least about 1 nucleotide, or at least about 2 nucleotides, or at least about 3 nucleotides, or at least about 4 nucleotides, or at least about 5 nucleotides, or at least about 6 nucleotides, or at least about 7 nucleotides, or at least about 8 nucleotides, or at least about 9 nucleotides, or at least about 10 nucleotides in length.

In some aspects, a 3′ overhang of a partially double-stranded identifier molecule is no more than 1, or no more than 2, or no more than 3, or no more than 4, or no more than 5, or no more than 6, or no more than 7, or no more than 8, or no more than 9, or no more than 10 nucleotides, or no more than 11 nucleotides, or no more than 12 nucleotides, or no more than 13 nucleotide, or no more than 14 nucleotides, or no more than 15 nucleotides, or no more than 16 nucleotides, or no more than 17 nucleotides, or no more than 18 nucleotides, or no more than 19 nucleotides, or no more than 20 nucleotides in length.

In some aspects, a 3′ overhang of a partially double-stranded identifier molecule can be 1 nucleotide in length. In some aspects, a 3′ overhang of a partially double-stranded identifier molecule can be 1 nucleotide in length, wherein the 1 nucleotide is an adenine or a thymine. In some aspects, a 3′ overhang of a partially double-stranded identifier molecule can be 1 nucleotide in length, wherein the 1 nucleotide is an adenine. In some aspects, a 3′ overhang of a partially double-stranded identifier molecule can be 1 nucleotide in length, wherein the 1 nucleotide is a thymine. In some aspects, a 3′ overhang of a partially double-stranded identifier molecule can be 1 nucleotide in length, wherein the 1 nucleotide is a guanine or a cytosine. In some aspects, a 3′ overhang of a partially double-stranded identifier molecule can be 1 nucleotide in length, wherein the 1 nucleotide is a guanine. In some aspects, a 3′ overhang of a partially double-stranded identifier molecule can be 1 nucleotide in length, wherein the 1 nucleotide is a cytosine.

In some aspects, a 5′ overhang of a partially double-stranded identifier molecule can be 1 nucleotide in length. In some aspects, a 5′ overhang of a partially double-stranded identifier molecule can be 1 nucleotide in length, wherein the 1 nucleotide is an adenine or a thymine. In some aspects, a 5′ overhang of a partially double-stranded identifier molecule can be 1 nucleotide in length, wherein the 1 nucleotide is an adenine. In some aspects, a 5′ overhang of a partially double-stranded identifier molecule can be 1 nucleotide in length, wherein the 1 nucleotide is a thymine. In some aspects, a 5′ overhang of a partially double-stranded identifier molecule can be 1 nucleotide in length, wherein the 1 nucleotide is a guanine or a thymine. In some aspects, a 5′ overhang of a partially double-stranded identifier molecule can be 1 nucleotide in length, wherein the 1 nucleotide is a guanine. In some aspects, a 5′ overhang of a partially double-stranded identifier molecule can be 1 nucleotide in length, wherein the 1 nucleotide is a cytosine.

In some aspects, the double-stranded region of a partially double-stranded identifier molecule can be at least about 1 nucleotide in length, at least about 2 nucleotides in length, or at least about 3 nucleotides in length, or at least about 4 nucleotides in length, or at least about 5 nucleotides in length, or at least about 6 nucleotides in length, or at least about 7 nucleotides in length, or at least about 8 nucleotides in length, or at least about 9 nucleotides in length, or at least about 10 nucleotides in length, or at least about 11 nucleotides in length, or at least about 12 nucleotides in length, or at least about 13 nucleotides in length, or at least about 14 nucleotides in length, or at least about 15 nucleotides in length, or at least about 16 nucleotides in length, or at least about 17 nucleotides in length, or at least about 18 nucleotides in length, or at least about 19 nucleotides in length, or at least about 20 nucleotides in length in length, or at least about 21 nucleotides in length, or at least about 22 nucleotides in length, or at least about 23 nucleotides in length, or at least about 24 nucleotides in length, or at least about 25 nucleotides in length, or at least about 26 nucleotides in length, or at least about 27 nucleotides in length, or at least about 28 nucleotides in length, or at least about 29 nucleotides in length, or at least about 30 nucleotides in length.

In some aspects, the double-stranded region of a partially double-stranded identifier molecule can be about 1 nucleotide in length, about 2 nucleotides in length, or about 3 nucleotides in length, or about 4 nucleotides in length, or about 5 nucleotides in length, or about 6 nucleotides in length, or about 7 nucleotides in length, or about 8 nucleotides in length, or about 9 nucleotides in length, or about 10 nucleotides in length, or about 11 nucleotides in length, or about 12 nucleotides in length, or about 13 nucleotides in length, or about 14 nucleotides in length, or about 15 nucleotides in length, or about 16 nucleotides in length, or about 17 nucleotides in length, or about 18 nucleotides in length, or about 19 nucleotides in length, or about 20 nucleotides in length in length, or about 21 nucleotides in length, or about 22 nucleotides in length, or about 23 nucleotides in length, or about 24 nucleotides in length, or about 25 nucleotides in length, or about 26 nucleotides in length, or about 27 nucleotides in length, or about 28 nucleotides in length, or about 29 nucleotides in length, or about 30 nucleotides in length. In some aspects, the double-stranded region of a partially double-stranded identifier molecule is about 9 nucleotides in length. In some aspects, the double-stranded region of a partially double-stranded identifier molecule is about 10 nucleotides in length. In some aspects, the double-stranded region of a partially double-stranded identifier molecule is about 11 nucleotides in length. In some aspects, the double-stranded region of a partially double-stranded identifier molecule is about 12 nucleotides in length. In some aspects, the double-stranded region of a partially double-stranded identifier molecule is about 19 nucleotides in length. In some aspects, the double-stranded region of a partially double-stranded identifier molecule is about 20 nucleotides in length. In some aspects, the double-stranded region of a partially double-stranded identifier molecule is about 21 nucleotides in length. In some aspects, the double-stranded region of a partially double-stranded identifier molecule is about 22 nucleotides in length.

In some aspects, an identifier sequence of a partially double-stranded identifier molecule can span the entire double-stranded region of a partially-double stranded identifier molecule. In some aspects, an identifier sequence of a partially double-stranded identifier molecule can span a portion of the double-stranded region of a partially-double stranded identifier molecule.

Accordingly, an identifier sequence can be at least about 1 nucleotide in length, at least about 2 nucleotides in length, or at least about 3 nucleotides in length, or at least about 4 nucleotides in length, or at least about 5 nucleotides in length, or at least about 6 nucleotides in length, or at least about 7 nucleotides in length, or at least about 8 nucleotides in length, or at least about 9 nucleotides in length, or at least about 10 nucleotides in length, or at least about 11 nucleotides in length, or at least about 12 nucleotides in length, or at least about 13 nucleotides in length, or at least about 14 nucleotides in length, or at least about 15 nucleotides in length, or at least about 16 nucleotides in length, or at least about 17 nucleotides in length, or at least about 18 nucleotides in length, or at least about 19 nucleotides in length, or at least about 20 nucleotides in length in length, or at least about 21 nucleotides in length, or at least about 22 nucleotides in length, or at least about 23 nucleotides in length, or at least about 24 nucleotides in length, or at least about 25 nucleotides in length, or at least about 26 nucleotides in length, or at least about 27 nucleotides in length, or at least about 28 nucleotides in length, or at least about 29 nucleotides in length, or at least about 30 nucleotides in length.

Accordingly, an identifier sequence be about 1 nucleotide in length, about 2 nucleotides in length, or about 3 nucleotides in length, or about 4 nucleotides in length, or about 5 nucleotides in length, or about 6 nucleotides in length, or about 7 nucleotides in length, or about 8 nucleotides in length, or about 9 nucleotides in length, or about 10 nucleotides in length, or about 11 nucleotides in length, or about 12 nucleotides in length, or about 13 nucleotides in length, or about 14 nucleotides in length, or about 15 nucleotides in length, or about 16 nucleotides in length, or about 17 nucleotides in length, or about 18 nucleotides in length, or about 19 nucleotides in length, or about 20 nucleotides in length in length, or about 21 nucleotides in length, or about 22 nucleotides in length, or about 23 nucleotides in length, or about 24 nucleotides in length, or about 25 nucleotides in length, or about 26 nucleotides in length, or about 27 nucleotides in length, or about 28 nucleotides in length, or about 29 nucleotides in length, or about 30 nucleotides in length. In some aspects, an identifier sequence is about 9 nucleotides in length. In some aspects, an identifier sequence is about 10 nucleotides in length. In some aspects, an identifier sequence is about 11 nucleotides in length. In some aspects, an identifier sequence is about 12 nucleotides in length. In some aspects, an identifier sequence is about 19 nucleotides in length. In some aspects, an identifier sequence is about 20 nucleotides in length. In some aspects, an identifier sequence is about 21 nucleotides in length. In some aspects, an identifier sequence is about 22 nucleotides in length.

Exemplary identifier sequences are shown in Table 1. Accordingly, an identifier sequence can comprise any of the sequences in Table 1, or a reverse complement thereof.

TABLE 1
Exemplary Identifier Sequences

| Strand #1 | SEQ ID NO | Strand #2 | SEQ ID NO |
|---|---|---|---|
| AACCGCGGCT | 1 | GCCGCGGTTAGA | 13 |
| GGTTATAACT | 2 | GTTATAACCAGA | 14 |
| CCAAGTCCGT | 3 | CGGACTTGGAGA | 15 |
| TTGGACTTCT | 4 | GAAGTCCAAAGA | 16 |
| CAGTGGATCT | 5 | GATCCACTGAGA | 17 |
| TGACAAGCGT | 6 | CGCTTGTCAAGA | 18 |
| CGAGATATGT | 7 | CATATCTCGAGA | 19 |
| TAGAGCGCGT | 8 | CGCGCTCTAAGA | 20 |
| AACCTGTTGT | 9 | CAACAGGTTAGA | 21 |
| GGTTCACCGT | 10 | CGGTGAACCAGA | 22 |
| CATTGTTGCT | 11 | GCAACAATGAGA | 23 |
| TGCCACCAGT | 12 | CTGGTGGCAAGA | 24 |
| Strand #1 | SEQ ID NO | Strand #2 | SEQ ID NO |
| CTAGCTTGCT | 25 | GCAAGCTAGAGA | 37 |
| TCGATCCAGT | 26 | CTGGATCGAAGA | 38 |
| CCTGAACTGT | 27 | CAGTTCAGGAGA | 39 |
| TTCAGGTCGT | 28 | CGACCTGAAAGA | 40 |
| AGTAGAGAGT | 29 | CTCTCTACTAGA | 41 |
| GACGAGAGCT | 30 | GCTCTCGTCAGA | 42 |
| CTCTGCCTGT | 31 | CAGGCAGAGAGA | 43 |
| TCTCATTCGT | 32 | CGAATGAGAAGA | 44 |
| ACGCCGCAGT | 33 | CTGCGGCGTAGA | 45 |
| GTATTATGCT | 34 | GCATAATACAGA | 46 |
| GATAGATCGT | 35 | CGATCTATCAGA | 47 |
| AGCGAGCTGT | 36 | CAGCTCGCTAGA | 48 |
| Strand #1 | SEQ ID NO | Strand #2 | SEQ ID NO |
| AGACTTGGCT | 49 | GCCAAGTCTAGA | 61 |
| GAGTCCAAGT | 50 | CTTGGACTCAGA | 62 |
| CTTAAGCCGT | 51 | CGGCTTAAGAGA | 63 |
| TCCGGATTCT | 52 | GAATCCGGAAGA | 64 |
| CTGTATTACT | 53 | GTAATACAGAGA | 65 |
| TCACGCCGCT | 54 | GCGGCGTGAAGA | 66 |
| CAGTTCCGCT | 55 | GCGGAACTGAGA | 67 |
| TGACCTTAGT | 56 | CTAAGGTCAAGA | 68 |
| CTAGGCAAGT | 57 | CTTGCCTAGAGA | 69 |
| TCGAATGGCT | 58 | GCCATTCGAAGA | 70 |
| CTTAGTGTCT | 59 | GACACTAAGAGA | 71 |
| TCCGACACGT | 60 | CGTGTCGGAAGA | 72 |
| Strand #1 | SEQ ID NO | Strand #2 | SEQ ID NO |
| ACTTACATGT | 73 | CATGTAAGTAGA | 85 |
| GTCCGTGCGT | 74 | CGCACGGACAGA | 86 |
| AAGGTACCGT | 75 | CGGTACCTTAGA | 87 |
| GGAACGTTCT | 76 | GAACGTTCCAGA | 88 |
| AATTCTGCGT | 77 | CGCAGAATTAGA | 89 |
| GGCCTCATGT | 78 | CATGAGGCCAGA | 90 |
| AACAGGAAGT | 79 | CTTCCTGTTAGA | 91 |
| GGTGAAGGCT | 80 | GCCTTCACCAGA | 92 |
| CCTGTGGCGT | 81 | CGCCACAGGAGA | 93 |
| TTCACAATGT | 82 | CATTGTGAAAGA | 94 |
| ACACGAGTGT | 83 | CACTCGTGTAGA | 95 |
| GTGTAGACGT | 84 | CGTCTACACAGA | 96 |
| Strand #1 | SEQ ID NO | Strand #2 | SEQ ID NO |
| AGCGCTAGCGT | 97 | CGCTAGCGCTAGA | 109 |
| GATATCGATCT | 98 | GATCGATATCAGA | 110 |
| CGCAGACGCGT | 99 | CGCGTCTGCGAGA | 111 |
| TATGAGTATCT | 100 | GATACTCATAAGA | 112 |
| AGGTGCGTCGT | 101 | CGACGCACCTAGA | 113 |
| GAACATACGCT | 102 | GCGTATGTTCAGA | 114 |
| ATCTTAGTCGT | 103 | CGACTAAGATAGA | 115 |
| GCTCCGACTGT | 104 | CAGTCGGAGCAGA | 116 |
| ATACCAAGCGT | 105 | CGCTTGGTATAGA | 117 |
| GCGTTGGATCT | 106 | GATCCAACGCAGA | 118 |
| CTTCACGGCGT | 107 | CGCCGTGAAGAGA | 119 |
| TCCTGTAATCT | 108 | GATTACAGGAAGA | 120 |
| Strand #1 | SEQ ID NO | Strand #2 | SEQ ID NO |
| ACATAGCGCGT | 121 | CGCGCTATGTAGA | 133 |
| GTGCGATATCT | 122 | GATATCGCACAGA | 134 |
| CCAACAGATGT | 123 | CATCTGTTGGAGA | 135 |
| TTGGTGAGCGT | 124 | CGCTCACCAAAGA | 136 |
| CGCGGTTCTCT | 125 | GAGAACCGCGAGA | 137 |
| TATAACCTCGT | 126 | CGAGGTTATAAGA | 138 |
| AGAATGCCTGT | 127 | CAGGCATTCTAGA | 139 |
| GAGGCATTCGT | 128 | CGAATGCCTCAGA | 140 |
| CCTCGGTATCT | 129 | GATACCGAGGAGA | 141 |
| TTCTAACGCGT | 130 | CGCGTTAGAAAGA | 142 |
| ATGAGGCTCGT | 131 | CGAGCCTCATAGA | 143 |
| GCAGAATCTGT | 132 | CAGATTCTGCAGA | 144 |
| Strand #1 | SEQ ID NO | Strand #2 | SEQ ID NO |
| AAGGATGATCT | 145 | GATCATCCTTAGA | 157 |
| GGAAGCAGCGT | 146 | CGCTGCTTCCAGA | 158 |
| TCGTGACCGCT | 147 | GCGGTCACGAAGA | 159 |
| CTACAGTTCGT | 148 | CGAACTGTAGAGA | 160 |
| ATATTCACGCT | 149 | GCGTGAATATAGA | 161 |
| GCGCCTGTACT | 150 | GTACAGGCGCAGA | 162 |
| CACTACGATGT | 151 | CATCGTAGTGAGA | 163 |
| TGTCGTAGCGT | 152 | CGCTACGACAAGA | 164 |
| ACCACTTATGT | 153 | CATAAGTGGTAGA | 165 |
| GTTGTCCGCGT | 154 | CGCGGACAACAGA | 166 |
| ATCCATATCGT | 155 | CGATATGGATAGA | 167 |
| GCTTGCGCTCT | 156 | GAGCGCAAGCAGA | 168 |
| Strand #1 | SEQ ID NO | Strand #2 | SEQ ID NO |
| ACTCTATGCGT | 169 | CGCATAGAGTAGA | 181 |
| GTCTCGCATCT | 170 | GATGCGAGACAGA | 182 |
| AAGACGTCTGT | 171 | CAGACGTCTTAGA | 183 |
| GGAGTACTCGT | 172 | CGAGTACTCCAGA | 184 |
| ACCGGCCATGT | 173 | CATGGCCGGTAGA | 185 |
| GTTAATTGCGT | 174 | CGCAATTAACAGA | 186 |
| AGTATCTTCGT | 175 | CGAAGATACTAGA | 187 |
| GACGCTCCTGT | 176 | CAGGAGCGTCAGA | 188 |
| CATGCCATAGT | 177 | CTATGGCATGAGA | 189 |
| TGCATTGCGCT | 178 | GCGCAATGCAAGA | 190 |
| ATTGGAACGCT | 179 | GCGTTCCAATAGA | 191 |
| GCCAAGGTCGT | 180 | CGACCTTGGCAGA | 192 |

The present disclosure provides pluralities of partially double-stranded identifier molecules.

The present disclosure provides pluralities of partially double-stranded identifier molecules, wherein the plurality comprises at least about one, or at least about two, or at least about three, or at least about four, or at least about five, or at least about six, or at least about seven, or at least about eight, or at least about nine, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50, or at least about 51, or at least about 52, or at least about 53, or at least about 54, or at least about 55, or at least about 56, or at least about 57, or at least about 58, or at least about 59, or at least about 60, or at least about 61, or at least about 62, or at least about 63, or at least about 64, or at least about 65, or at least about 66, or at least about 67, or at least about 68, or at least about 69, or at least about 70, or at least about 71, or at least about 72, or at least about 73, or at least about 74, or at least about 75, or at least about 76, or at least about 77, or at least about 78, or at least about 79, or at least about 80, or at least about 81, or at least about 82, or at least about 83, or at least about 84, or at least about 85, or at least about 86, or at least about 87, or at least about 88, or at least about 89, or at least about 90, or at least about 91, or at least about 92, or at least about 93, or at least about 94, or at least about 95, or at least about 96, or at least about 97, or at least about 98, or at least about 99, or at least about 100 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides pluralities of partially double-stranded identifier molecules, wherein the plurality comprises about one, or about two, or about three, or about four, or about five, or about six, or about seven, or about eight, or about nine, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or about 65, or about 66, or about 67, or about 68, or about 69, or about 70, or about 71, or about 72, or about 73, or about 74, or about 75, or about 76, or about 77, or about 78, or about 79, or about 80, or about 81, or about 82, or about 83, or about 84, or about 85, or about 86, or about 87, or about 88, or about 89, or about 90, or about 91, or about 92, or about 93, or about 94, or about 95, or about 96, or about 97, or about 98, or about 99, or about 100 species of partially double-stranded identifier molecules, wherein each species of partially double-stranded identifier molecules comprises an identifier sequence that is different from the identifier sequence of any other species of partially double-stranded identifier molecules in the plurality.

In some aspects of the pluralities of partially double-stranded identifier molecules, each of the species of partially double-stranded identifier molecules can be present in the same amount, or different species of partially double-stranded identifier molecules can be present in different amounts.

The present disclosure provides a plurality of partially double-stranded identifier molecules, wherein the plurality comprises at least about 12 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises at least about 24 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises at least about 48 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises at least about 96 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises about 12 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises about 24 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises about 48 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises about 96 species of partially double-stranded identifier molecules, wherein each species of partially double-stranded identifier molecules comprises an identifier sequence that is different from the identifier sequence of any other species of partially double-stranded identifier molecules in the plurality.

In some aspects of the pluralities of partially double-stranded identifier molecules of the present disclosure, the identifier sequence of one species of partially double-stranded identifier molecules have a hamming distance of at least about two, or at least about three, or at least about four, or at least about five, or at least about six, or at least about seven, or at least about eight, or at least about nine, or at least about ten to any other identifier sequence of any other species of partially double-stranded identifier molecules in the plurality. In some aspects of the pluralities of partially double-stranded identifier molecules of the present disclosure, the identifier sequence of one species of partially double-stranded identifier molecules have a hamming distance of about two, or about three, or about four, or about five, or about six, or about seven, or about eight, or about nine, or about ten to any other identifier sequence of any other species of partially double-stranded identifier molecules in the plurality.

As would be appreciated by the skilled artisan, the “hamming distance” between two identifier sequences, identifier sequence x and identifier sequence y, corresponds to the number of changes that would need to be made in identifier sequence x to transform identifier sequence x into identifier sequence y, or vice versa.

Partially Double-Stranded Adapter Molecules

The present disclosure provides partially double-stranded adapter molecules. Partially double-stranded adapter molecules are nucleic acid molecules comprising at least one double-stranded region, at least three single stranded regions. In some aspects, a partially double-stranded adapter molecule is a nucleic acid molecule comprising one double-stranded region and three single stranded regions.

In some aspects a partially double-stranded adapter molecule comprises DNA. In some aspects, a partially double-stranded adapter molecule comprises RNA. In some aspects, a partially double-stranded adapter molecule can comprise XNA. In some aspects, a partially double-stranded adapter molecule comprises any combination of DNA, RNA and XNA.

In some aspects, a partially double-stranded adapter molecule can comprise one overhang. The overhang can be a 3′ overhang or a 5′ overhang.

In some aspects of the compositions of the present disclosure a 5′ overhang of a partially double-stranded adapter molecule can be about 1 nucleotide in length, or about 2 nucleotides in length, or about 3 nucleotides in length, or about 4 nucleotides in length, or about 5 nucleotides in length. In some aspects of the compositions of the present disclosure a 5′ overhang can be at least about 1 nucleotide, or at least about 2 nucleotides, or at least about 3 nucleotides, or at least about 4 nucleotides, or at least about 5 nucleotides, or at least about 6 nucleotides, or at least about 7 nucleotides, or at least about 8 nucleotides, or at least about 9 nucleotides, or at least about 10 nucleotides in length.

In some aspects, a 5′ overhang of a partially double-stranded adapter molecule is no more than 1, or no more than 2, or no more than 3, or no more than 4, or no more than 5, or no more than 6, or no more than 7, or no more than 8, or no more than 9, or no more than 10 nucleotides, or no more than 11 nucleotides, or no more than 12 nucleotides, or no more than 13 nucleotide, or no more than 14 nucleotides, or no more than 15 nucleotides, or no more than 16 nucleotides, or no more than 17 nucleotides, or no more than 18 nucleotides, or no more than 19 nucleotides, or no more than 20 nucleotides in length.

In some aspects of the compositions of the present disclosure a 3′ overhang of a partially double-stranded adapter molecule can be about 1 nucleotide in length, or about 2 nucleotides in length, or about 3 nucleotides in length, or about 4 nucleotides in length, or about 5 nucleotides in length. In some aspects of the compositions of the present disclosure a 3′ overhang can be at least about 1 nucleotide, or at least about 2 nucleotides, or at least about 3 nucleotides, or at least about 4 nucleotides, or at least about 5 nucleotides, or at least about 6 nucleotides, or at least about 7 nucleotides, or at least about 8 nucleotides, or at least about 9 nucleotides, or at least about 10 nucleotides in length.

In some aspects, a 3′ overhang of a partially double-stranded adapter molecule is no more than 1, or no more than 2, or no more than 3, or no more than 4, or no more than 5, or no more than 6, or no more than 7, or no more than 8, or no more than 9, or no more than 10 nucleotides, or no more than 11 nucleotides, or no more than 12 nucleotides, or no more than 13 nucleotide, or no more than 14 nucleotides, or no more than 15 nucleotides, or no more than 16 nucleotides, or no more than 17 nucleotides, or no more than 18 nucleotides, or no more than 19 nucleotides, or no more than 20 nucleotides in length.

In some aspects, a 3′ overhang of a partially double-stranded adapter molecule can be 1 nucleotide in length. In some aspects, a 3′ overhang of a partially double-stranded adapter molecule can be 1 nucleotide in length, wherein the 1 nucleotide is an adenine or a thymine. In some aspects, a 3′ overhang of a partially double-stranded adapter molecule can be 1 nucleotide in length, wherein the 1 nucleotide is an adenine. In some aspects, a 3′ overhang of a partially double-stranded adapter molecule can be 1 nucleotide in length, wherein the 1 nucleotide is a thymine. In some aspects, a 3′ overhang of a partially double-stranded adapter molecule can be 1 nucleotide in length, wherein the 1 nucleotide is a guanosine or a cytosine. In some aspects, a 3′ overhang of a partially double-stranded adapter molecule can be 1 nucleotide in length, wherein the 1 nucleotide is a guanosine. In some aspects, a 3′ overhang of a partially double-stranded adapter molecule can be 1 nucleotide in length, wherein the 1 nucleotide is a cytidine.

In some aspects, a 5′ overhang of a partially double-stranded adapter molecule can be 1 nucleotide in length. In some aspects, a 5′ overhang of a partially double-stranded adapter molecule can be 1 nucleotide in length, wherein the 1 nucleotide is an adenine or a thymine. In some aspects, a 5′ overhang of a partially double-stranded adapter molecule can be 1 nucleotide in length, wherein the 1 nucleotide is an adenine. In some aspects, a 5′ overhang of a partially double-stranded adapter molecule can be 1 nucleotide in length, wherein the 1 nucleotide is a thymine. In some aspects, a 5′ overhang of a partially double-stranded adapter molecule can be 1 nucleotide in length, wherein the 1 nucleotide is a guanosine or a cytosine. In some aspects, a 5′ overhang of a partially double-stranded adapter molecule can be 1 nucleotide in length, wherein the 1 nucleotide is a guanosine. In some aspects, a 5′ overhang of a partially double-stranded adapter molecule can be 1 nucleotide in length, wherein the 1 nucleotide is a cytidine.

In some aspects, a partially double-stranded adapter molecule can comprise a single-stranded arm. As would be appreciated by the skilled artisan, an “arm”, in the context of a partially double-stranded nucleic acid molecule refers to a single-stranded region of a partially double-stranded nucleic acid molecule located at a terminus of the partially double-stranded nucleic acid for which there is a corresponding single-stranded region located directly on the opposite strand. In some aspects, a single-stranded arm can be a single-stranded 5′ arm. In some aspects, a single-stranded arm can be a single-stranded 3′ arm. FIG. 3 shows both single-stranded 5′ arms and single-stranded 3′ arms in exemplary partially double-stranded adapter molecules of the present disclosure.

In some aspects, a single-stranded 5′ arm and/or single-stranded 3′ arm can be at least about 1 nucleotide in length, at least about 2 nucleotides in length, or at least about 3 nucleotides in length, or at least about 4 nucleotides in length, or at least about 5 nucleotides in length, or at least about 6 nucleotides in length, or at least about 7 nucleotides in length, or at least about 8 nucleotides in length, or at least about 9 nucleotides in length, or at least about 10 nucleotides in length, or at least about 11 nucleotides in length, or at least about 12 nucleotides in length, or at least about 13 nucleotides in length, or at least about 14 nucleotides in length, or at least about 15 nucleotides in length, or at least about 16 nucleotides in length, or at least about 17 nucleotides in length, or at least about 18 nucleotides in length, or at least about 19 nucleotides in length, or at least about 20 nucleotides in length in length, or at least about 21 nucleotides in length, or at least about 22 nucleotides in length, or at least about 23 nucleotides in length, or at least about 24 nucleotides in length, or at least about 25 nucleotides in length, or at least about 26 nucleotides in length, or at least about 27 nucleotides in length, or at least about 28 nucleotides in length, or at least about 29 nucleotides in length, or at least about 30 nucleotides in length.

In some aspects, a single-stranded 5′ arm and/or single-stranded 3′ arm can be about 1 nucleotide in length, about 2 nucleotides in length, or about 3 nucleotides in length, or about 4 nucleotides in length, or about 5 nucleotides in length, or about 6 nucleotides in length, or about 7 nucleotides in length, or about 8 nucleotides in length, or about 9 nucleotides in length, or about 10 nucleotides in length, or about 11 nucleotides in length, or about 12 nucleotides in length, or about 13 nucleotides in length, or about 14 nucleotides in length, or about 15 nucleotides in length, or about 16 nucleotides in length, or about 17 nucleotides in length, or about 18 nucleotides in length, or about 19 nucleotides in length, or about 20 nucleotides in length in length, or about 21 nucleotides in length, or about 22 nucleotides in length, or about 23 nucleotides in length, or about 24 nucleotides in length, or about 25 nucleotides in length, or about 26 nucleotides in length, or about 27 nucleotides in length, or about 28 nucleotides in length, or about 29 nucleotides in length, or about 30 nucleotides in length.

A single-stranded 5′ arm and/or single-stranded 3′ arm can comprise an amplification primer binding site that hybridizes to an amplification primer.

As would be appreciated by the skilled artisan, an amplification primer binding site is a nucleic acid sequence that is capable of being bound by a primer suitable for priming an amplification reaction using a nucleic acid polymerase. As would be appreciated by the skilled artisan, these amplification primer binding sites can be used to generate sequencing libraries using techniques that are standard in the art and well-known to the skilled artisan.

In some aspects, a partially double-stranded adapter molecule can comprise an identifier sequence, as is described above. In some aspects, an identifier sequence located in a partially double-stranded adapter molecule can be located in a double-stranded region of the partially double-stranded adapter molecule. In some aspects, an identifier sequence of a partially double-stranded adapter molecule can span the entire double-stranded region of a partially-double stranded adapter molecule. In some aspects, an identifier sequence of a partially double-stranded adapter molecule can span a region of the double-stranded region of a partially-double stranded adapter molecule.

In some aspects, the double-stranded region of a partially double-stranded adapter molecule can be at least about 1 nucleotide in length, at least about 2 nucleotides in length, or at least about 3 nucleotides in length, or at least about 4 nucleotides in length, or at least about 5 nucleotides in length, or at least about 6 nucleotides in length, or at least about 7 nucleotides in length, or at least about 8 nucleotides in length, or at least about 9 nucleotides in length, or at least about 10 nucleotides in length, or at least about 11 nucleotides in length, or at least about 12 nucleotides in length, or at least about 13 nucleotides in length, or at least about 14 nucleotides in length, or at least about 15 nucleotides in length, or at least about 16 nucleotides in length, or at least about 17 nucleotides in length, or at least about 18 nucleotides in length, or at least about 19 nucleotides in length, or at least about 20 nucleotides in length in length, or at least about 21 nucleotides in length, or at least about 22 nucleotides in length, or at least about 23 nucleotides in length, or at least about 24 nucleotides in length, or at least about 25 nucleotides in length, or at least about 26 nucleotides in length, or at least about 27 nucleotides in length, or at least about 28 nucleotides in length, or at least about 29 nucleotides in length, or at least about 30 nucleotides in length.

In some aspects, the double-stranded region of a partially double-stranded adapter molecule can be about 1 nucleotide in length, about 2 nucleotides in length, or about 3 nucleotides in length, or about 4 nucleotides in length, or about 5 nucleotides in length, or about 6 nucleotides in length, or about 7 nucleotides in length, or about 8 nucleotides in length, or about 9 nucleotides in length, or about 10 nucleotides in length, or about 11 nucleotides in length, or about 12 nucleotides in length, or about 13 nucleotides in length, or about 14 nucleotides in length, or about 15 nucleotides in length, or about 16 nucleotides in length, or about 17 nucleotides in length, or about 18 nucleotides in length, or about 19 nucleotides in length, or about 20 nucleotides in length in length, or about 21 nucleotides in length, or about 22 nucleotides in length, or about 23 nucleotides in length, or about 24 nucleotides in length, or about 25 nucleotides in length, or about 26 nucleotides in length, or about 27 nucleotides in length, or about 28 nucleotides in length, or about 29 nucleotides in length, or about 30 nucleotides in length. In some aspects, the double-stranded region of a partially double-stranded adapter molecule is about 9 nucleotides in length. In some aspects, the double-stranded region of a partially double-stranded adapter molecule is about 10 nucleotides in length. In some aspects, the double-stranded region of a partially double-stranded adapter molecule is about 11 nucleotides in length. In some aspects, the double-stranded region of a partially double-stranded adapter molecule is about 12 nucleotides in length. In some aspects, the double-stranded region of a partially double-stranded adapter molecule is about 19 nucleotides in length. In some aspects, the double-stranded region of a partially double-stranded adapter molecule is about 20 nucleotides in length. In some aspects, the double-stranded region of a partially double-stranded adapter molecule is about 21 nucleotides in length. In some aspects, the double-stranded region of a partially double-stranded adapter molecule is about 22 nucleotides in length.

In some aspects, the partially double-stranded adapter molecules of the present disclosure comprise a single-stranded 5′ arm, a single-stranded 3′ arm, a double-stranded region and a 3′ overhang. An exemplary schematic of the preceding partially double-stranded adapter molecule is shown in the top panel of FIG. 3.

In some aspects, the partially double-stranded adapter molecules of the present disclosure comprise a single-stranded 5′ arm, a single-stranded 3′ arm, a double-stranded region and a 5′ overhang. An exemplary schematic of the preceding partially double-stranded adapter molecule is shown in the top panel of FIG. 3.

In the preceding partially double-stranded adapter molecules, the single-stranded 5′ arm, the single-stranded 3′ arm, or both the single-stranded 5′ arm and the single-stranded 3′ arm can comprise amplification primer binding sites.

In the preceding partially double-stranded adapter molecules, the double-stranded region can comprise an identifier sequence.

The present disclosure provides pluralities of partially double-stranded adapter molecules.

The present disclosure provides pluralities of partially double-stranded adapter molecules, wherein the plurality comprises at least about one, or at least about two, or at least about three, or at least about four, or at least about five, or at least about six, or at least about seven, or at least about eight, or at least about nine, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50, or at least about 51, or at least about 52, or at least about 53, or at least about 54, or at least about 55, or at least about 56, or at least about 57, or at least about 58, or at least about 59, or at least about 60, or at least about 61, or at least about 62, or at least about 63, or at least about 64, or at least about 65, or at least about 66, or at least about 67, or at least about 68, or at least about 69, or at least about 70, or at least about 71, or at least about 72, or at least about 73, or at least about 74, or at least about 75, or at least about 76, or at least about 77, or at least about 78, or at least about 79, or at least about 80, or at least about 81, or at least about 82, or at least about 83, or at least about 84, or at least about 85, or at least about 86, or at least about 87, or at least about 88, or at least about 89, or at least about 90, or at least about 91, or at least about 92, or at least about 93, or at least about 94, or at least about 95, or at least about 96, or at least about 97, or at least about 98, or at least about 99, or at least about 100 species of partially double-stranded adapter molecules, wherein each species of partially double-stranded adapter molecules comprises an identifier sequence that is different from the identifier sequence of any other species of partially double-stranded adapter molecules in the plurality.

The present disclosure provides pluralities of partially double-stranded adapter molecules, wherein the plurality comprises about one, or about two, or about three, or about four, or about five, or about six, or about seven, or about eight, or about nine, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or about 65, or about 66, or about 67, or about 68, or about 69, or about 70, or about 71, or about 72, or about 73, or about 74, or about 75, or about 76, or about 77, or about 78, or about 79, or about 80, or about 81, or about 82, or about 83, or about 84, or about 85, or about 86, or about 87, or about 88, or about 89, or about 90, or about 91, or about 92, or about 93, or about 94, or about 95, or about 96, or about 97, or about 98, or about 99, or about 100 species of partially double-stranded adapter molecules, wherein each species of partially double-stranded adapter molecules comprises an identifier sequence that is different from the identifier sequence of any other species of partially double-stranded adapter molecules in the plurality.

In some aspects of the pluralities of partially double-stranded adapter molecules, each of the species of partially double-stranded adapter molecules can be present in the same amount, or different species of partially double-stranded adapter molecules can be present in different amounts.

In some aspects, any of the partially double-stranded nucleic acid molecules described herein, including partially double-stranded identifier molecules and partially double-stranded adapter molecules can comprise at least one modified nucleic acid. In some aspects, a modified nucleic acid can comprise methylated cytidine. In some aspects, a modified nucleic acid can comprise 5mC (5-methylcytosine), 5hmC (5-hydromethylcytosine), 5fC (5-formylcytosine), 3 mA (3-methyladenine), 5-fU (5-formyluridine), 5-hmU (5-hydroxymethyluridine), 5-hoU (5-hydroxyuridine), 7mG (7-methylguanine), 8oxoG (8-oxo-7,8-dihydroguanine), AP (apurinic/apyrimidinic sites), CPDs (Cyclobutane pyrimidine dimers), dI (deoxyinosine), dR5P (deoxyribose 5′-phosphate), dU (deoxyuridine), dX (deoxyxanthosine), PA (3′-phospho-α,β-unsaturated aldehyde), rN (ribonucleotides), Tg (Thymine Glycol), TT (TT dimer) and/or Mismatches including AP:A (apurinic/apyrimidinic site base paired with adenine), DHT:A (5,6-dihydrothymine base paired with an adenine), 5-hmU:A (5-hydroxymethyluracil base paired with an adenine), 5-hmU:G (5-hydroxymethyluracil base paired with a guanine), I:T (inosine base paired with a thymine), 6-MeA:T (6-methyladenine base paired with a thymine), 8-OG:C (8-oxoguanine base paired with a cytosine), 8-OG:G (8-oxoguanine base paired with a guanine), U:A (uridine base paired with an adenine) or U:G (uridine base paired with a guanine) or any combination thereof.

Kits

The present disclosure provides kits comprising the compositions of the present disclosure. These compositions include, but are not limited to, the any of the partially double-stranded nucleic acid molecules described herein, including, but not limited to, partially double-stranded identifier molecules and partially double-stranded adapter molecules; any of the pluralities of partially double-stranded nucleic acid molecules, including, but not limited to pluralities of partially double-stranded identifier molecules and pluralities of partially double-stranded adapter molecules.

Accordingly, the present disclosure provides a kit comprising a plurality of partially double-stranded identifier molecules, wherein the plurality comprises at least about one, or at least about two, or at least about three, or at least about four, or at least about five, or at least about six, or at least about seven, or at least about eight, or at least about nine, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50, or at least about 51, or at least about 52, or at least about 53, or at least about 54, or at least about 55, or at least about 56, or at least about 57, or at least about 58, or at least about 59, or at least about 60, or at least about 61, or at least about 62, or at least about 63, or at least about 64, or at least about 65, or at least about 66, or at least about 67, or at least about 68, or at least about 69, or at least about 70, or at least about 71, or at least about 72, or at least about 73, or at least about 74, or at least about 75, or at least about 76, or at least about 77, or at least about 78, or at least about 79, or at least about 80, or at least about 81, or at least about 82, or at least about 83, or at least about 84, or at least about 85, or at least about 86, or at least about 87, or at least about 88, or at least about 89, or at least about 90, or at least about 91, or at least about 92, or at least about 93, or at least about 94, or at least about 95, or at least about 96, or at least about 97, or at least about 98, or at least about 99, or at least about 100 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises about one, or about two, or about three, or about four, or about five, or about six, or about seven, or about eight, or about nine, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or about 65, or about 66, or about 67, or about 68, or about 69, or about 70, or about 71, or about 72, or about 73, or about 74, or about 75, or about 76, or about 77, or about 78, or about 79, or about 80, or about 81, or about 82, or about 83, or about 84, or about 85, or about 86, or about 87, or about 88, or about 89, or about 90, or about 91, or about 92, or about 93, or about 94, or about 95, or about 96, or about 97, or about 98, or about 99, or about 100 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises at least about 12 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises at least about 24 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a plurality of partially double-stranded identifier molecules, wherein the plurality comprises at least about 48 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises at least about 96 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises about 12 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises about 24 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises about 48 species of partially double-stranded identifier molecules, wherein each species of partially double-stranded identifier molecules comprises an identifier sequence that is different from the identifier sequence of any other species of partially double-stranded identifier molecules in the plurality.

The present disclosure provides a kit comprising a plurality of partially double-stranded identifier molecules, wherein the plurality comprises about 96 species of partially double-stranded identifier molecules, wherein each species of partially double-stranded identifier molecules comprises an identifier sequence that is different from the identifier sequence of any other species of partially double-stranded identifier molecules in the plurality.

In some aspects of the kits of the present disclosure, each species of partially double-stranded identifier molecules is kept physically separate from other species of partially double-stranded identifier molecules.

In a non-limiting example, physical separation can be accomplished by enclosing each species of partially double-stranded identifier molecules in a separate container (e.g. different wells in a microplate, different sample tubes, etc.). By physically separate each species of partially double-stranded identifier molecules, the kit allows the user to optimize the number of barcode combinations to be used with each sample that is to be analyzed using the kit.

The kits of the present disclosure can further comprise a plurality of partially double-stranded adapter molecules. In some aspects, the plurality of partially double-stranded adapter molecules comprises at least about one, or at least about two, or at least about three, or at least about four, or at least about five, or at least about six, or at least about seven, or at least about eight, or at least about nine, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50, or at least about 51, or at least about 52, or at least about 53, or at least about 54, or at least about 55, or at least about 56, or at least about 57, or at least about 58, or at least about 59, or at least about 60, or at least about 61, or at least about 62, or at least about 63, or at least about 64, or at least about 65, or at least about 66, or at least about 67, or at least about 68, or at least about 69, or at least about 70, or at least about 71, or at least about 72, or at least about 73, or at least about 74, or at least about 75, or at least about 76, or at least about 77, or at least about 78, or at least about 79, or at least about 80, or at least about 81, or at least about 82, or at least about 83, or at least about 84, or at least about 85, or at least about 86, or at least about 87, or at least about 88, or at least about 89, or at least about 90, or at least about 91, or at least about 92, or at least about 93, or at least about 94, or at least about 95, or at least about 96, or at least about 97, or at least about 98, or at least about 99, or at least about 100 species of partially double-stranded adapter molecules, wherein each species of partially double-stranded adapter molecules comprises an identifier sequence that is different from the identifier sequence of any other species of partially double-stranded adapter molecules in the plurality. In some aspects, the plurality of partially double-stranded adapter molecules comprises about one, or about two, or about three, or about four, or about five, or about six, or about seven, or about eight, or about nine, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or about 65, or about 66, or about 67, or about 68, or about 69, or about 70, or about 71, or about 72, or about 73, or about 74, or about 75, or about 76, or about 77, or about 78, or about 79, or about 80, or about 81, or about 82, or about 83, or about 84, or about 85, or about 86, or about 87, or about 88, or about 89, or about 90, or about 91, or about 92, or about 93, or about 94, or about 95, or about 96, or about 97, or about 98, or about 99, or about 100 species of partially double-stranded adapter molecules, wherein each species of partially double-stranded adapter molecules comprises an identifier sequence that is different from the identifier sequence of any other species of partially double-stranded adapter molecules in the plurality.

The kits of the present disclosure can further comprise a plurality of enzymes to mediate end-repair on double-stranded DNA molecules. Such pluralities of enzymes are well-known to the skilled artisan and include, but are not limited to, pluralities comprising DNA polymerases (e.g. T4 DNA polymerase), klenow fragments, polynucleotide kinases (e.g. T4 polynucleotide kinase) or any combination thereof.

The kits of the present disclosure can further comprise a plurality of reagents suitable for the purification of nucleic acid molecules. Such pluralities of reagents are well-known to the skilled artisan.

The kits of the present disclosure can further comprise at least one DNA ligase. The DNA ligase can be any DNA ligase known in the art, including but not limited to, T4 DNA ligase, T7 DNA ligase or any other DNA ligase known in the art.

The kits of the present disclosure can further comprise a plurality of amplification primers that bind to one or more of the amplification primer binding sites located on partially double-stranded adapter molecules.

The kits of the present disclosure can further comprise at least one DNA polymerase. In some aspects, the at least one DNA polymerase is able to catalyze amplification via the amplification primers that bind to one or more of the amplification primer binding sites located on partially double-stranded adapter molecules.

The kits of the present disclosure can further comprise written instructions for the performance of the methods of the present disclosure.

Methods of Sequencing Target Nucleic Acids

The present disclosure provides methods for sequencing target nucleic acids. The sequencing methods, compositions and kits of the present disclosure exhibit superior properties as compared to existing NGS methods that use pre-pooled unique molecular identifies (UMIs).

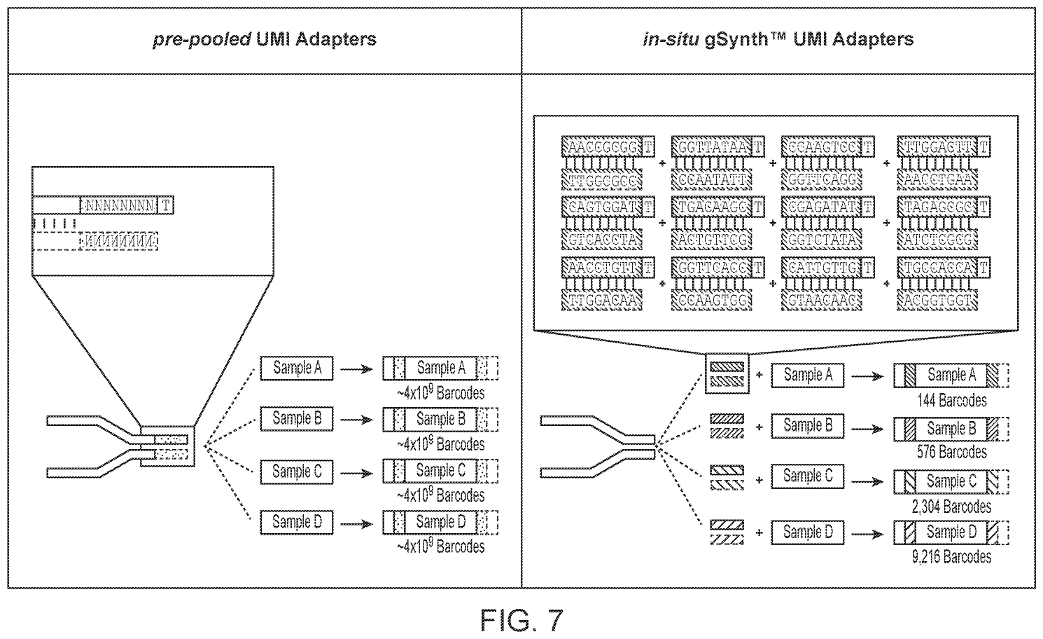

As would be appreciated by the skilled artisan, existing NGS methods rely on the expensive synthesis of an entire adapter molecule per each barcode sequence that is to be used in an experiment. Moreover, in existing pre-pooled barcoded adapter products, there is no flexibility in the number and the length of barcodes that are used for individual samples. Moreover, existing pooled barcodes increase the risk of cross-talk and have a maximum hamming distance of one, so error-correction of barcodes is not possible. In contrast to existing methods, the sequencing composition, kits and methods of the present disclosure are more cost-effective, as only a single adapter needs to be synthesized for use with all identifier sequences. Moreover, the compositions, kits and methods of the present disclosure allow for a fully customizable number of barcodes to be used for each sample. That is, the number of barcodes used for a particular sample can be optimized for that particular sample type and/or experimental objective. The compositions, kits and methods of the present disclosure allow for all identifier sequences to remain completely independent, reducing the risk of crosstalk. Finally, the identifier sequences of the compositions, kits and methods of the present disclosure having hamming distances of at least two, allowing for error-correction and increased barcode fidelity. FIG. 7 shows a schematic comparison between existing next generation sequencing barcode compositions and methods and the compositions and the methods of the present disclosure.

In the methods of the present disclosure, the ligation of a partially double-stranded identifier molecule of the present disclosure to each of a transcript in a plurality of target nucleic acids results in the creation of a UMI sequence that is ligated to that transcript. In other words, the transcript becomes tagged with a combination of two identifier sequences through the ligation of partially double-stranded identifier molecules to each end. For example, in a method wherein four species of partially double-stranded identifier molecules are used, say identifier x, identifier y, identifier w, and identifier z, the random ligation of one of partially double-stranded identifier molecules to each end of the transcript could create one of 16 UMIs, as shown in Table 2.

TABLE 2
Exemplary UMI Combinations:

Identifier x + Identifier x
Identifier y + Identifier y
Identifier w + Identifier w
Identifier z + Identifier z
Identifier x + Identifier y
Identifier x + Identifier w
Identifier x + Identifier z
Identifier y + Identifier x
Identifier y + Identifier w
Identifier y + Identifier z
Identifier z + Identifier x
Identifier z + Identifier w
Identifier z + Identifier y
Identifier w + Identifier x
Identifier w + Identifier z
Identifier w + Identifier y

These UMI sequences that are created by the ligation steps of the methods of the present disclosure can then be used in analysis using methods standard in the art, including, but not limited to, error correction, consensus sequence creation, etc.

In some aspects, target nucleic acids are double-stranded nucleic acid molecules.

In some aspects, target nucleic acids can comprise DNA, RNA or a combination of DNA and RNA.

Target nucleic acids can be derived from any source, including, but not limited to any biological sample. Target nucleic acids can be extracted from biological samples using techniques that are standard in the art. After extraction from a biological samples, target nucleic acids and be processed using techniques that are standard in the art prior to being subjected to the methods of the present disclosure. These processing methods can include, but are not limited to, fragmentation, reverse transcription, end-repair or any other nucleic acid processing technique known in the art. In aspects wherein RNA is extracted from a biological sample, the RNA can be reverse transcribed into DNA prior to being subjected to the methods of the present disclosure.

The sequencing methods of the present disclosure can comprise: a) ligating a first partially double-stranded identifier molecule to one end of a target nucleic acid; b) ligating a second partially double-stranded identifier molecule to the other end of the target nucleic acid; c) ligating a first partially double-stranded adapter molecule to the first partially double-stranded identifier molecule; and d) ligating a second partially double-stranded adapter molecule to the second partially double-stranded identifier molecule.

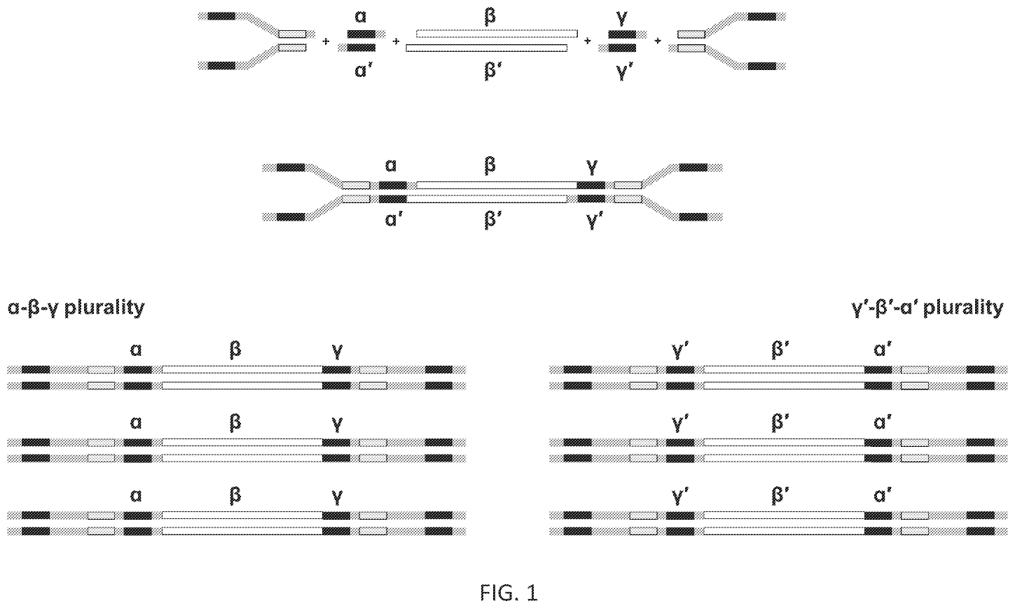

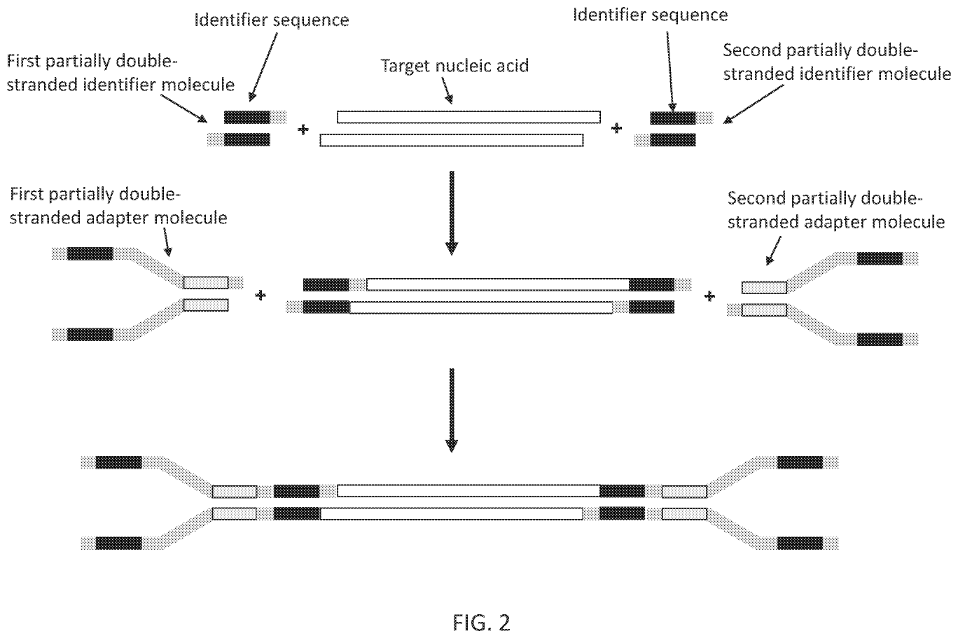

In some aspects of the preceding method, steps (a) and (b) can be performed sequentially. In some aspects of the preceding method, steps (a) and (b) can be performed concurrently. In some aspects of the preceding method, steps (c) and (d) can be performed sequentially. In some aspects of the preceding method, steps (c) and (d) can be performed concurrently. A non-limiting example of the preceding method is shown in FIG. 1 and FIG. 2. In the top panel of FIG. 2, a first partially double-stranded identifier molecule and a second partially double-stranded identifier molecule are ligated to the ends of a target nucleic acid. In the middle panel, a first partially double-stranded adapter molecule and a second partially double-stranded adapter molecule are ligated to the first partially double-stranded identifier molecule and the second partially double-stranded identifier molecule, respectively.

The present disclosure provides methods of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides methods of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about two species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise each of the at least four combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides methods of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about 12 species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise each of the at least 144 combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides methods of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about 24 species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise each of the at least 576 combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides methods of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about 96 species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise each of the at least 9,216 combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides a method of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about two species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% of the at least four combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides a method of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about 12 species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% of the at least 144 combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides a method of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about 24 species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% of the at least 576 combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

The present disclosure provides a method of sequencing a plurality of double-stranded target nucleic acids comprising: a) contacting the plurality of double-stranded target nucleic acids with a plurality of partially double-stranded identifier molecules of the present disclosure and at least one ligase such that a partially double-stranded identifier molecule is ligated to each end of the double-stranded target nucleic acids in the plurality of double-stranded target nucleic acids, wherein the plurality of partially double-stranded identifier molecules of the present disclosure comprises at least about 96 species of the partially double-stranded identifier molecules, wherein each species of partially double-stranded identifier molecules comprises an identifier sequence that is different from the identifier sequence of any other species of partially double-stranded identifier molecules in the plurality, wherein the ligation products comprise at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% of the at least 9,216 combinations of two species of partially double-stranded identifier molecules; b) contacting the products of step (a) with a plurality of partially double-stranded adapter molecules of the present disclosure and at least one ligase such that a partially double-stranded adapter molecule is ligated to each end of the products of step (a); and c) sequencing the products of step (b).

In some aspects of the preceding methods, sequencing can be performed using any sequencing method known in the art, including, but not limited to, next generation sequencing methods, sequencing-by-synthesis methods, sequencing by ligation methods, single-molecule real-time sequencing methods, ion semiconductor sequencing methods, pyrosequencing methods, combinatorial probe anchor synthesis sequencing methods, nanopore sequencing methods, genanpsys sequencing methods, sanger sequencing methods or any other sequencing method known in the art.

In some aspects of the preceding methods, the methods can further comprise after step (b) and prior to step (c), constructing a sequencing library using the products of step (b). The sequencing library can be constructed using standard library construction techniques known in the art. These library construction techniques can comprise amplifying the products of step (b) by contacting the products of step (b) with amplification primers that bind to amplification primer binding sites and at least one polymerase. The amplification can comprise the introduction of sequencing adapters that are suitable for use in the sequencing method of choice.

In some aspects, the library construction techniques can comprise nucleic acid purification techniques that are known in the art.

In some aspects, the preceding method can further comprise determining the abundance and/or the identity of specific transcripts in the plurality of double-stranded target nucleic acids using the sequencing data generated in step (c). As would be appreciated by the skilled artisan, the identifier sequences of the ligated partially double-stranded identifier molecules can be used in the analysis of the sequencing data to determine the abundance of specific transcripts by allowing the skilled artisan to correct various errors introduced during the sequencing process (including, but not limited to, amplification errors) using methods standard in the art. As would be appreciated by the skilled artisan, the identifier sequences of the ligated partially double-stranded identifier molecules can be used in the analysis of the sequencing data to determine the identity of specific transcripts by allowing the skilled artisan to create consensus sequences using methods standard in the art.

In some aspects, determining the abundance and/or identity of specific transcripts in the plurality of double-stranded target nucleic acids using the sequencing data generated in step (c) can comprise grouping the sequencing reads obtained in step (c) by the ligated identifier sequences in the sequencing reads.

In some aspects, determining the abundance and/or identity of specific transcripts in the plurality of double-stranded target nucleic acids using the sequence data generated in step (c) can comprise grouping the sequencing reads obtained in step (c) by the specific genomic sequence that the sequencing reads most likely correspond to.

In some aspects, determining the abundance and/or identity of specific transcripts in the plurality of double-stranded target nucleic acids using the sequencing data generated in step (c) can comprise first grouping the sequencing reads obtained in step (c) by the ligated identifier sequences in the sequencing reads and then further grouping by the specific genomic sequence that the sequencing reads most likely correspond to.

Without wishing to be bound by theory, for existing pre-pooled, degenerate UMIs, the number of UMIs available should be larger than the number of molecules present within the initial sample. This ensures each molecule gets a unique UMI. This approach leads to a large majority of UMIs containing only a single read. With a minimum requirement of at least two reads per UMI, to generate a consensus sequence, the UMIs containing only a single read are discarded. The inability to produce a consensus read for a large majority of the available UMIs, means that very high sequencing depths are required for each region of interest. These limitations are overcome by the methods of the present disclosure by: 1) giving the user the ability to modulate the number of discrete UMIs used for the initial sample; 2) using the sequence from the region of interest as a UMI itself.

In some aspects of the methods of the present disclosure, determining the abundance and/or identity of specific transcripts in the plurality of double-stranded target nucleic acids using the sequencing data generated in step (c) can comprising grouping together aligned sequencing reads based on their absolute alignment to a reference sequence (e.g. a known genomic sequence). In some aspects, these aligned sequencing reads can then be further grouped based on their similarity from the reference sequence. In some aspects, the aligned sequencing reads can then further be sub-divided by their UMIs. Because the methods of the present disclosure allow the number of initial UMIs to be modulated, the number of sequencing reads per UMI can be modulated to have on average at least two sequencing reads per UMLI. Optimization of the number of reads per UMI, allows the majority of reads (and therefore UMIs) to produce usable consensus reads, therefore reducing the coverage required per region of interest.

Accordingly, in the methods of the present disclosure, the number of species of partially double-stranded identifier molecules that are used can be selected such that there is on average at least about two, or at least about three, or at least about four, or at least about five, or at least about six, or at least about seven, or at least about eight, or at least about nine, or at least about ten sequencing reads per UMI (i.e. combination of two species of double-stranded identifier molecules ligated onto a target transcript).

Accordingly, in the methods of the present disclosure, the number of species of partially double-stranded identifier molecules that are used can be selected such that there is at least about two, or at least about three, or at least about four, or at least about five, or at least about six, or at least about seven, or at least about eight, or at least about nine, or at least about ten sequencing reads per UMI (i.e. combination of two species of double-stranded identifier molecules ligated onto a target transcript).

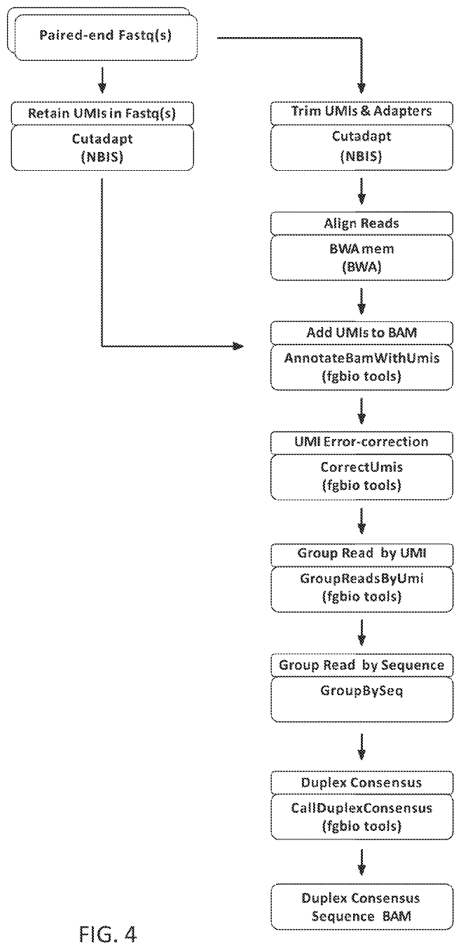

An exemplary sequencing data analysis workflow is shown in FIG. 4.

In some aspects, determining the abundance and/or identify of specific transcripts in the plurality of double-stranded target nucleic acids can comprise determining the frequency of one or more mutations in a specific transcript in the plurality of double-stranded target nucleic acid. Mutations can include, but are not limited to one or more substitutions, one or more deletions, one or more insertions, one or more deletion-insertions, one or more duplications, one or more inversions, one or more repeat expansions or any combination thereof.

In some aspects of the preceding methods, the number of species of partially double-stranded identifier molecules in the plurality of partially double-stranded identifier molecules can be optimized to provide the appropriate sensitivity for the specific sequencing application. Without wishing to be bound by theory, in sequencing applications with the aim of identifying and/or quantifying rare mutations, the number of species can be increased to provide an increased number of possible barcode combinations.

The present disclosure provides methods for sequencing collections of double-stranded nucleic acid molecules using randomly paired adapter DNA constructs that together create combinatorial barcodes. Barcodes are used to identify and quantify individual variant molecules within a complex DNA sample. The method comprises the steps: (a) affixing individual identifier molecules (containing discrete hemi-barcodes) to both ends of double stranded DNA fragments, while also affixing either an individual adapter molecule or an individual identifier adapter molecule onto the identifier molecule, to create a double stranded DNA fragment that contains a pair of identifier molecules and a pair of adapter molecules or a pair of identifier adapter molecules; (b) a single identifier molecule contains a sequence, that allows specific sticky-end ligation and is compatible with the adapter molecule or identifier adapter molecule. A second sequence that allows distinct, specific sticky-end ligation and is compatible with the target DNA fragments; (c) a single identifier molecule also contains a degenerate, semi-degenerate or discrete (non-degenerate) nucleic acid sequence which creates a relatively unique barcode; (d) The single adapter molecule contains a double stranded hybridized region, a sequence, that allows specific sticky-end ligation compatible with a sticky-end sequence on the identifier molecule, a single-stranded 5′ arm, and a single stranded 3′ arm, with further identifier molecules being affixed to the DNA-adapter fragment via amplification; (e) The single identifier adapter molecule contains a double stranded hybridized region, a sequence, that allows specific sticky-end ligation compatible with a sticky-end sequence on the identifier molecule, a single-stranded 5′ arm with a single stranded identifier, and a single stranded 3′ arm with a single stranded identifier.

The present disclosure provides a plurality of molecules is obtained by: (a) amplification of a single strand or both strands of the target DNA fragments prior to applying adapter molecules to the double stranded DNA targets; (b) amplification of a single strand or both strands of the target DNA-adapter product subsequent to applying adapter molecules to the double stranded DNA targets; (c) a combination of amplifications of either a single strand or both strands of the target DNA fragments prior to and/or subsequent to applying adapter molecules with index identifiers to the double stranded DNA targets; (d) Sequencing the amplified DNA-adapter products, thereby obtaining the association of each DNA target molecules with their corresponding barcodes to allow for downstream process such as error correction of barcodes, determining plurality of reads per barcode, determining of and correcting for errors associated with the sample preparation and sequencing of the target DNA molecules, determining true identities of target DNA sequences from potential false identities.

### Exemplary Embodiments

Embodiment 1. A composition comprising:
a plurality of partially double-stranded identifier molecules; and a plurality of partially double-stranded adapter molecules.

Embodiment 2. The composition of embodiment 1, wherein the partially double-stranded identifier molecules comprise nucleic acid sequences about 11-20 nucleotides in length.

Embodiment 3. The composition of any of the preceding embodiments, wherein the partially double-stranded identifier molecules comprise at least one 5′ overhang.

Embodiment 4. The composition of embodiment 3, wherein the partially double-stranded identifier molecules comprise two 5′ overhangs.

Embodiment 5. The composition of embodiment 3, wherein the 5′ overhang(s) is/are about 3 to about 5 nucleotides in length.

Embodiment 6. The composition of any one of embodiments 3-5, wherein at least one 5′ overhang is capable of ligation to the partially double-stranded adapter molecules.

Embodiment 7. The composition of any one of embodiments 3-6, wherein at least one 5′ overhang is capable of ligation to a target nucleic acid obtained from a biological sample.

Embodiment 8. The composition of any of the preceding embodiments, wherein the partially double-stranded adapter molecules comprise a double-stranded hybridized region.

Embodiment 9. The composition of any of the preceding embodiments, wherein the partially double-stranded adapter molecules comprise at least one overhang.

Embodiment 10. The composition of embodiment 10, wherein the overhang is capable of ligation to the partially double-stranded identifier molecules.

Embodiment 11. The composition of any of the preceding embodiments, wherein the partially double-stranded adapter molecules comprise a single-stranded 5′ arm.

Embodiment 12. The composition of any of the preceding embodiments, wherein the partially double-stranded adapter molecules comprise a single-stranded 3′ arm.

Embodiment 13. A kit comprising the composition of any of the preceding embodiments.

Embodiment 14. The kit of embodiment 13, further comprising a plurality of enzymes to mediate end-repair on double stranded DNA targets.

Embodiment 15. The kit of embodiment 13 or embodiment 14, further comprising a DNA ligase to mediate ligation of the adapter molecule or identifier adapter molecule and identifier molecule.

Embodiment 16. The kit of any one of embodiments 13-15, further comprising a set of primers suitable for the amplification of the DNA-adapter molecules.

Embodiment 17. The kit of any one of embodiments 13-16, further comprising a DNA polymerase to mediate the amplification of the DNA-adapter molecules.

Embodiment 18. The kit of any one of embodiments 13-17, further comprising reagents suitable for the purification of the end-repaired double stranded DNA targets and/or ligated DNA-adapter molecules and/or amplified DNA-adapter molecules.

Embodiment 19. The kit of any one of embodiments 13-18, further comprising buffers suitable to perform the appropriate enzymatic and purification steps.

Embodiment 20. The kit of any one of embodiments 13-19, further comprising written instructions.

Embodiment 21. A method for sequencing collections of double-stranded nucleic acid molecules using randomly paired adapter DNA constructs that together create combinatorial barcodes, wherein barcodes are used to identify and quantify individual variant molecules within a complex DNA sample, the method comprising:
a) affixing at least one partially double-stranded identifier molecule (containing discrete hemi-barcodes) to both ends of a target DNA fragment, wherein the identifier molecule comprises a discrete hemi-barcode,
b) affixing either at least one adapter molecule or identifier adapter molecule onto the identifier molecules, thereby producing a double stranded DNA fragment comprising a pair of identifier molecules and a pair of adapter molecules or identifier adapter molecules,

Embodiment 22. The method of embodiment 21, wherein the at least one identifier molecule comprises a degenerate, semi-degenerate or discrete (non-degenerate) nucleic acid sequence which creates a unique barcode.

Embodiment 23. The method of embodiment 21 or embodiment 22, wherein the at least one adapter molecule comprises a double stranded hybridized region, a sequence, that allows specific sticky-end ligation compatible with a sticky-end sequence on the at least one identifier molecule, a single-stranded 5′ arm, and a single stranded 3′ arm.

Embodiment 24. The method of any one of embodiments 21-23, the method further comprising affixing additional identifier molecules to the target DNA-adapter fragment via amplification.

Embodiment 25. The method of any one of embodiments 21-24, wherein the at least one identifier adapter molecule comprises a double stranded hybridized region, a sequence, which allows specific sticky-end ligation compatible with a sticky-end sequence on the at least one identifier molecule, a single-stranded 5′ arm with a single stranded identifier, and a single stranded 3′ arm with a single stranded identifier.

Embodiment 26. The method of any one of embodiments 21-25, the method further comprising amplifying a single strand or both strands of the target DNA fragments prior to applying adapter molecules to the double stranded DNA targets.

Embodiment 27. The method of any one of embodiments 21-26, the method further comprising amplifying a single strand or both strands of the target DNA-identifier product subsequent to applying adapter molecules to the double stranded DNA targets.

Embodiment 28. The method of any one of embodiments 21-27, the method further comprising sequencing the amplified DNA-adapter products, thereby obtaining the association of each DNA target molecules with their corresponding barcodes.

Embodiment 29. The method of embodiment 28, wherein association of each DNA molecules with their corresponding barcodes allow for at least one downstream process, wherein the downstream process is selected from error correction of barcodes, determining plurality of reads per barcode, determining of and correcting for errors associated with the sample preparation and sequencing of the target DNA molecules, determining true identities of target DNA sequences from potential false identities.

Embodiment 30. The method of any one of embodiments 21-29, wherein the at least one adapter molecule or identifier adapter molecule comprises a primer binding site.

Embodiment 31. The method of embodiment 30, wherein the primer binding site comprises a nucleotide sequence that permits for the linear or exponential amplification.

Embodiment 32. The method of any one of embodiments 21-30, wherein the at least one identifier molecule contains an error correctable, discrete hemi-barcodes.

Embodiment 33. A partially double-stranded identifier molecule comprising:
a double-stranded region; and
a first overhang.

Embodiment 34. The partially double-stranded identifier molecule of the embodiment 33, further comprising a second overhang.

Embodiment 35. The partially double-stranded identifier molecule of any one of embodiments 33-34, wherein the first and second overhangs are 5′ overhangs.

Embodiment 36. The partially double-stranded identifier molecule of any one of embodiments 33-34, wherein the first and second overhangs are 3′ overhangs.

Embodiment 37. The partially double-stranded identifier molecule of any one of embodiments 33-36, wherein the double-stranded region comprises an identifier sequence.

Embodiment 38. The partially double-stranded identifier molecule of embodiment 37, wherein the identifier sequence spans the entire double-stranded region.

Embodiment 39. The partially double-stranded identifier molecule of embodiment 37, wherein the identifier sequence spans a portion of the double-stranded region.

Embodiment 40. The partially double-stranded identifier molecule of any one of embodiments 37-39, wherein the identifier sequence is about 9 nucleotides in length.

Embodiment 41. The partially double-stranded identifier molecule of any one of embodiments 37-39, wherein the identifier sequence is about 10 nucleotides in length.

Embodiment 42. The partially double-stranded identifier molecule of any one of embodiments 37-39, wherein the identifier sequence is about 11 nucleotides in length.

Embodiment 43. The partially double-stranded identifier molecule of any one of embodiments 37-39, wherein the identifier sequence is about 12 nucleotides in length.

Embodiment 44. The partially double-stranded identifier molecule of any one of embodiments 37-39, wherein the identifier sequence is about 19 nucleotides in length.

Embodiment 45. The partially double-stranded identifier molecule of any one of embodiments 37-39, wherein the identifier sequence is about 20 nucleotides in length.

Embodiment 46. The partially double-stranded identifier molecule of any one of embodiments 37-39, wherein the identifier sequence is about 21 nucleotides in length.

Embodiment 47. The partially double-stranded identifier molecule of any one of embodiments 37-39, wherein the identifier sequence is about 22 nucleotides in length.

Embodiment 48. The partially double-stranded identifier molecule of any one of embodiments 33-47, wherein the first overhang is about 1 nucleotide in length.

Embodiment 49. The partially double-stranded identifier molecule of embodiment 48, wherein the first overhang is an adenine or a thymine.

Embodiment 50. The partially double-stranded identifier molecule of any one of embodiments 33-47, wherein the first overhang is about 2 nucleotides in length.

Embodiment 51. The partially double-stranded identifier molecule of any one of embodiments 33-47, wherein the first overhang is about 3 nucleotides in length.

Embodiment 52. The partially double-stranded identifier molecule of any one of embodiments 33-47, wherein the first overhang is about 4 nucleotides in length.

Embodiment 53. The partially double-stranded identifier molecule of any one of embodiments 33-47, wherein the first overhang is about 5 nucleotides in length.

Embodiment 54. The partially double-stranded identifier molecule of any one of embodiments 34-53, wherein the second overhang is about 1 nucleotide in length.

Embodiment 55. The partially double-stranded identifier molecule of embodiment 54, wherein the second overhang is an adenine or a thymine.

Embodiment 56. The partially double-stranded identifier molecule of any one of embodiments 34-53, wherein the second overhang is about 2 nucleotides in length.

Embodiment 57. The partially double-stranded identifier molecule of any one of embodiments 34-53, wherein the second overhang is about 3 nucleotides in length.

Embodiment 58. The partially double-stranded identifier molecule of any one of embodiments 34-53, wherein the second overhang is about 4 nucleotides in length.

Embodiment 59. The partially double-stranded identifier molecule of any one of embodiments 34-53, wherein the second overhang is about 5 nucleotides in length.

Embodiment 60. The partially double-stranded identifier molecule of any one of embodiments 33-59, wherein the partially double-stranded identifier molecule comprises DNA.

Embodiment 61. A plurality of the partially double-stranded identifier molecules of any one of embodiments 33-60, wherein the plurality comprises at least about 12 species of the partially double-stranded identifier molecules, wherein each species of partially double-stranded identifier molecules comprises an identifier sequence that is different from the identifier sequence of any other species of partially double-stranded identifier molecules in the plurality.

Embodiment 62. The plurality of embodiment 61, wherein the plurality comprises at least about 24 species of the partially double-stranded identifier molecules.

Embodiment 63. The plurality of embodiment 62, wherein the plurality comprises at least about 48 species of the partially double-stranded identifier molecules.

Embodiment 64. The plurality of embodiment 63, wherein the plurality comprises at least about 96 species of the partially double-stranded identifier molecules.

Embodiment 65. The plurality of any one of embodiments 61-64, wherein the identifier sequence of one species of partially double-stranded identifier molecules will have a hamming distance of at least about two to any other identifier sequence of any other species of partially double-stranded identifier molecules in the plurality.

Embodiment 66. A partially double-stranded adapter molecule comprising:
a double-stranded region;
an overhang;
a single-stranded 5′ arm; and
a single-stranded 3′ arm.

Embodiment 67. The partially double-stranded adapter molecule of embodiment 66, wherein the overhang is a 5′ overhang.

Embodiment 68. The partially double-stranded adapter molecule of embodiment 66, wherein the overhang is a 3′ overhang.

Embodiment 69. The partially double-stranded adapter molecule of any one of embodiments 66-68, wherein the overhang is about 1 nucleotide in length.

Embodiment 70. The partially double-stranded adapter molecule of embodiment 69, wherein the overhang is an adenine or a thymine.

Embodiment 71. The partially double-stranded adapter molecule of any one of embodiments 66-68, wherein the overhang is about 2 nucleotides in length.

Embodiment 72. The partially double-stranded adapter molecule of any one of embodiments 66-68, wherein the overhang is about 3 nucleotides in length.

Embodiment 73. The partially double-stranded adapter molecule of any one of embodiments 66-68, wherein the overhang is about 4 nucleotides in length.

Embodiment 74. The partially double-stranded adapter molecule of any one of embodiments 66-68, wherein the overhang is about 5 nucleotides in length.

Embodiment 75. The partially double-stranded adapter molecule of any one of embodiments 66-74, wherein the double-stranded region comprises an identifier sequence.

Embodiment 76. The partially double-stranded adapter molecule of embodiment 75, wherein the identifier sequence is about 9 nucleotides in length.

Embodiment 77. The partially double-stranded adapter molecule of embodiment 75, wherein the identifier sequence is about 10 nucleotides in length.

Embodiment 78. The partially double-stranded adapter molecule of embodiment 75, wherein the identifier sequence is about 11 nucleotides in length.

Embodiment 79. The partially double-stranded adapter molecule of embodiment 75, wherein the identifier sequence is about 12 nucleotides in length.

Embodiment 80. The partially double-stranded adapter molecule of embodiment 75, wherein the identifier sequence is about 19 nucleotides in length.

Embodiment 81. The partially double-stranded adapter molecule of embodiment 75, wherein the identifier sequence is about 20 nucleotides in length.

Embodiment 82. The partially double-stranded adapter molecule of embodiment 75, wherein the identifier sequence is about 21 nucleotides in length.

Embodiment 83. The partially double-stranded adapter molecule of embodiment 75, wherein the identifier sequence is about 22 nucleotides in length.

Embodiment 84. The partially double-stranded adapter molecule of any one of embodiments 66-83, wherein the single-stranded 5′ arm comprises at least one amplification primer binding site.

Embodiment 85. The partially double-stranded adapter molecule of any one of embodiments 66-84, wherein the single-stranded 3′ arm comprises at least one amplification primer binding site.

Embodiment 86. The partially double-stranded adapter molecule of any one of embodiments 66-85, wherein the partially double-stranded adapter molecule comprises DNA.

Embodiment 87. A plurality of the partially double-stranded adapter molecules of any one of embodiments 66-85, wherein the plurality comprises at least about 12 species of the partially double-stranded adapter molecules, wherein each species of partially double-stranded adapter molecules comprises an identifier sequence that is different from the identifier sequence of any other species of partially double-stranded adapter molecules in the plurality.

Embodiment 88. The plurality of embodiment 87, wherein the plurality comprises at least about 24 species of the partially double-stranded adapter molecules.

Embodiment 89. The plurality of embodiment 88, wherein the plurality comprises at least about 48 species of the partially double-stranded adapter molecules.

Embodiment 90. The plurality of embodiment 89, wherein the plurality comprises at least about 96 species of the partially double-stranded adapter molecules.

Embodiment 91. The plurality of any one of embodiments 87-90, wherein the identifier sequence of one species of partially double-stranded identifier molecules have a hamming distance of at least about two to any other identifier sequence of any other species of partially double-stranded identifier molecules in the plurality.

Embodiment 92. A kit comprising the plurality of any one of embodiments 61-65.

Embodiment 93. The kit of embodiment 92, further comprising the plurality of any one of embodiments 87-91.

Embodiment 94. The kit of embodiment 92 or 93, further comprising a plurality of enzymes to mediate end-repair on double-stranded.

Embodiment 95. The kit of any one of embodiments 92-94, further comprising a plurality of reagents for the purification of nucleic acid molecules.

Embodiment 96. The kit of any one of embodiments 92-95, further comprising at least one DNA polymerase.

Embodiment 97. The kit of any one of embodiments 92-96, further comprising a plurality of amplification primers.

Embodiment 98. The kit of embodiment 97, wherein the amplification primers in the plurality bind to the amplification primer binding sites present in the partially double-stranded adapter molecules.

Embodiment 99. The kit of any one of embodiments 92-98, further comprising at least one DNA ligase.

100. The kit of any one of embodiments 92-99, further comprising written instructions for the performance of one of the methods of the present disclosure.

Embodiment 101. A method of sequencing a plurality of double-stranded target nucleic acids comprising:
a) contacting the plurality of double-stranded target nucleic acids with the plurality of partially double-stranded identifier molecules of any one of embodiments 61-65 and at least one ligase such that a partially double-stranded identifier molecule is ligated to each end of the double-stranded target nucleic acids in the plurality of double-stranded target nucleic acids;
b) contacting the products of step (a) with a plurality of partially double-stranded adapter molecules of the present disclosure and at least one ligase such that a partially double-stranded adapter molecule is ligated to each end of the products of step (a); and
c) sequencing the products of step (b).

Embodiment 102. A method of sequencing a plurality of double-stranded target nucleic acids comprising:
a) contacting the plurality of double-stranded target nucleic acids with the plurality of partially double-stranded identifier molecules of any one of embodiments 61-65 and at least one ligase such that a partially double-stranded identifier molecule is ligated to each end of the double-stranded target nucleic acids in the plurality of double-stranded target nucleic acids, wherein the ligation products comprise each of the combinations of two species of partially double-stranded identifier molecules;
b) contacting the products of step (a) with a plurality of partially double-stranded adapter molecules of the present disclosure and at least one ligase such that a partially double-stranded adapter molecule is ligated to each end of the products of step (a); and
c) sequencing the products of step (b).

Embodiment 103. A method of sequencing a plurality of double-stranded target nucleic acids comprising:
a) contacting the plurality of double-stranded target nucleic acids with the plurality of partially double-stranded identifier molecules of any one of embodiments 61-65 and at least one ligase such that a partially double-stranded identifier molecule is ligated to each end of the double-stranded target nucleic acids in the plurality of double-stranded target nucleic acids, wherein the ligation products comprise at least 10% of the combinations of two species of partially double-stranded identifier molecules;
b) contacting the products of step (a) with a plurality of partially double-stranded adapter molecules of the present disclosure and at least one ligase such that a partially double-stranded adapter molecule is ligated to each end of the products of step (a); and
c) sequencing the products of step (b).

Embodiment 104. The method of embodiment 103, wherein the ligation products in step (a) comprise at least 20% of the combinations of two species of partially double-stranded identifier molecules.

Embodiment 105. The method of embodiment 104, wherein the ligation products in step (a) comprise at least 30% of the combinations of two species of partially double-stranded identifier molecules.

Embodiment 106. The method of embodiment 105, wherein the ligation products in step (a) comprise at least 40% of the combinations of two species of partially double-stranded identifier molecules.

Embodiment 107. The method of embodiment 106, wherein the ligation products in step (a) comprise at least 50% of the combinations of two species of partially double-stranded identifier molecules.

Embodiment 108. The method of embodiment 107, wherein the ligation products in step (a) comprise at least 60% of the combinations of two species of partially double-stranded identifier molecules.

Embodiment 109. The method of embodiment 108, wherein the ligation products in step (a) comprise at least 70% of the combinations of two species of partially double-stranded identifier molecules.

Embodiment 110. The method of embodiment 109, wherein the ligation products in step (a) comprise at least 80% of the combinations of two species of partially double-stranded identifier molecules.

Embodiment 111. The method of embodiment 110, wherein the ligation products in step (a) comprise at least 90% of the combinations of two species of partially double-stranded identifier molecules.

Embodiment 112. The method of any one of embodiments 101-111, the method further comprising after step (b) and prior to step (c), constructing a sequencing library using the products of step (b).

Embodiment 113. The method of any one of embodiments 101-112, the method further comprising after step (b) and prior to step (c), amplifying the products of step (b).

Embodiment 114. The method of embodiment 113, wherein amplifying the products of step (b) comprises contacting the products of step b with amplification primers that bind to amplification primer binding sites in the partially double-stranded adapter molecules and at least one polymerase.

Embodiment 115. The method of any one of embodiments 101-114, wherein the method further comprising determining the abundance and/or the identity of specific transcripts in the plurality of double-stranded target nucleic acids using the sequencing data generated in step (c).

Embodiment 116. The method of embodiment 115, wherein determining the abundance and/or the identity of specific transcripts in the plurality of double-stranded target nucleic acids using the sequencing data generated in step (c) comprises correcting for errors using the identifier sequences of the ligated partially double-stranded identifier molecules.

Embodiment 117. The method of embodiment 116, wherein the errors comprise amplification errors, sample preparation errors, sequencing errors or any combination thereof.

Embodiment 118. The method of any one of embodiments 115-117, wherein determining the abundance and/or the identity of specific transcripts in the plurality of double-stranded target nucleic acids using the sequencing data generated in step (c) comprises creating consensus sequences using identifier sequences of the ligated partially double-stranded identifier molecules.

Embodiment 119. The method of any one of embodiments 115-118, wherein determining the abundance and/or identity of specific transcripts in the plurality of double-stranded target nucleic acids using the sequencing data generated in step (c) comprises grouping the sequencing reads obtained in step (c) by the ligated identifier sequences in the sequencing reads.

Embodiment 120. The method of any one of embodiments 115-119, wherein determining the abundance and/or identity of specific transcripts in the plurality of double-stranded target nucleic acids using the sequence data generated in step (c) comprises grouping the sequencing reads obtained in step (c) by the specific genomic sequence that the sequencing reads most likely correspond to.

Embodiment 121. The method of any one of embodiments 115-120, wherein determining the abundance and/or identity of specific transcripts in the plurality of double-stranded target nucleic acids using the sequencing data generated in step (c) comprises first grouping the sequencing reads obtained in step (c) by the ligated identifier sequences in the sequencing reads and then further grouping by the specific genomic sequence that the sequencing reads most likely correspond to.

Embodiment 122. The method of any one of embodiments 115-121, wherein determining the abundance and/or identify of specific transcripts in the plurality of double-stranded target nucleic acids can comprise determining the frequency of one or more mutations in a specific transcript in the plurality of double-stranded target nucleic acid.

Embodiment 123. The method of embodiment 122, wherein the one or more mutations comprise one or more insertions, one or more deletion-insertions, one or more duplications, one or more inversions, one or more repeat expansions or any combination thereof.

Embodiment 124. The method of any one of embodiments 101-123, wherein the number of species of partially double-stranded identifier molecules in the plurality is selected such that there is on average at least about two sequencing reads for each UMI that is measured.

Embodiment 125. The method of any one of embodiments 101-124, wherein the number of species of partially double-stranded identifier molecules in the plurality is selected such that there at least about two sequencing reads for each UMI that is measured.

### EXAMPLES

### Example 1—Ligation of Partially Double-Stranded Identifier Molecules and Partially Double-Stranded Adapter Molecules of the Present Disclosure

The following is a non-limiting example of the ligation of partially double-stranded identifier molecules with varying overhang sizes and partially double-stranded adapter molecules of the present disclosure with varying overhang sizes and varying sizes of the double-strand regions to target nucleic acids using the methods of the present disclosure.

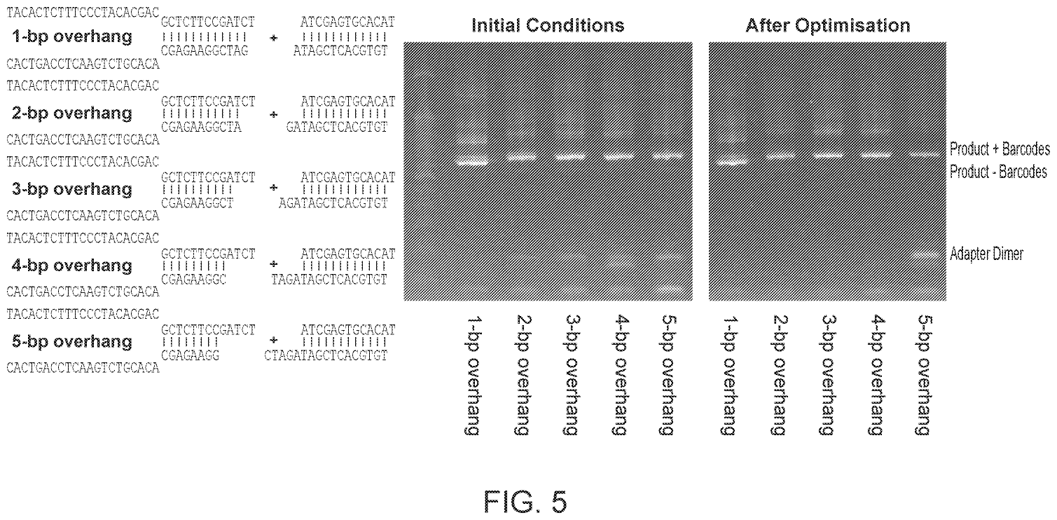

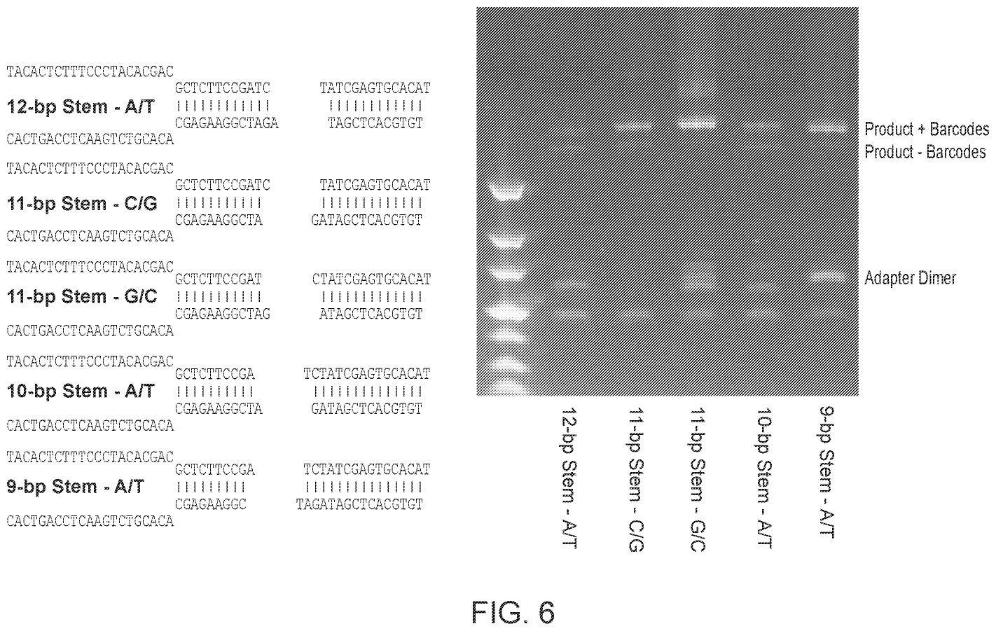

The double-stranded sequences of the partially double-stranded identifier molecules and partially double-stranded adapter molecules are shown below and in FIG. 5 and FIG. 6

Partially Double-Stranded Adapter Molecules with Varying Overhang Sizes

| | |
|---|---|
| Uni_Short | TACACTCTTTCCCTACACGACGCTCTTCCGATCT (SEQ ID NO: 193) |
| | \|\|\|\|\|\|\|\|\|\|\|\|\| |
| IDX_1 bp_Overhang | CACTGACCTCAAGTCTGCACACGAGAAGGCTAG (SEQ ID NO: 194) |
| Uni_Short | TACACTCTTTCCCTACACGACGCTCTTCCGATCT (SEQ ID NO: 193) |
| | \|\|\|\|\|\|\|\|\|\|\| |
| IDX_2bp_Overhang | CACTGACCTCAAGTCTGCACACGAGAAGGCTA (SEQ ID NO: 195) |
| Uni_Short | TACACTCTTTCCCTACACGACGCTCTTCCGATCT (SEQ ID NO: 193) |
| | \|\|\|\|\|\|\|\|\|\| |
| IDX_3 bp_Overhang | CACTGACCTCAAGTCTGCACACGAGAAGGCT (SEQ ID NO: 196) |
| Uni Short | TACACTCTTTCCCTACACGACGCTCTTCCGATCT (SEQ ID NO: 193) |
| | \|\|\|\|\|\|\|\|\| |
| IDX_4 bp_Overhang | CACTGACCTCAAGTCTGCACACGAGAAGGC (SEQ ID NO: 197) |
| Uni Short | TACACTCTTTCCCTACACGACGCTCTTCCGATCT (SEQ ID NO: 193) |
| | \|\|\|\|\|\|\|\| |
| IDX_5 bp_Overhang | CACTGACCTCAAGTCTGCACACGAGAAGG (SEQ ID NO: 198) |

Partially Double-Stranded Identifier Molecules with Varying Overhang Sizes

BC01a_Overhang
(SEQ ID NO: 199)
ATCGAGTGCACAT
BC01b_1 bp_Overhang
(SEQ ID NO: 200)
ATAGCTCACGTGT
BC01a_Overhang
(SEQ ID NO: 199)
ATCGAGTGCACAT
BC01b_2 bp_Overhang
(SEQ ID NO: 201)
GATAGCTCACGTGT
BC01a_Overhang
(SEQ ID NO: 199)
ATCGAGTGCACAT
BC01b_3 bp_Overhang
(SEQ ID NO: 202)
AGATAGCTCACGTGT
BC01a_Overhang
(SEQ ID NO: 199)
ATCGAGTGCACAT
BC01b_4 bp_Overhang
(SEQ ID NO: 203)
TAGATAGCTCACGTGT
BC01a_Overhang
(SEQ ID NO: 199)
ATCGAGTGCACAT
BC01b_5 bp_Overhang
(SEQ ID NO: 204)
CTAGATAGCTCACGTGT

Primers for 300 bp PCR Product

pUC19_300 bp_Frw_primer CGAGGGAGCTTCCAGGGGGAAAC (SEQ ID NO: 205)
pUC19 300 bp Rev primer TTGGGCGCTCTTCCGCTTCCTC (SEQ ID NO: 206)

Partially Double-Stranded Adapter Molecules with Varying Sizes of the Double-Stranded Region and Corresponding Partially Double-Stranded Identifier Molecules

| | |
|---|---|
| 12 bp_Stem_A-T_UNI | TACACTCTTTCCCTACACGACGCTCTTCCGATC (SEQ ID NO: 207) |
| | \|\|\|\|\|\|\|\|\|\|\|\| |
| 12 bp_Stem_A-T_IDX | CACTGACCTCAAGTCTGCACACGAGAAGGCTAGA (SEQ ID NO: 208) |
| 12 bp_Stem_A-T_BC01a | TATCGAGTGCACAT (SEQ ID NO: 209) |
| | \|\|\|\|\|\|\|\|\|\|\|\| |
| 12 bp_Stem_A-T_BC01b | TAGCTCACGTGT (SEQ ID NO: 210) |
| 11-bp_Stem_C-G_UNI | TACACTCTTTCCCTACACGACGCTCTTCCGATC (SEQ ID NO: 211) |
| | \|\|\|\|\|\|\|\|\|\|\| |
| 11-bp_Stem_C-G_IDX | CACTGACCTCAAGTCTGCACACGAGAAGGCTA (SEQ ID NO: 212) |
| 11-bp_Stem_C-G_BC01a | TATCGAGTGCACAT (SEQ ID NO: 209) |
| | \|\|\|\|\|\|\|\|\|\|\|\|\| |
| 11-bp_Stem_C-G_BC01b | GATAGCTCACGTGT (SEQ ID NO: 213) |
| 11-bp_Stem_G-C_UNI | TACACTCTTTCCCTACACGACGCTCTTCCGAT (SEQ ID NO: 214) |
| | \|\|\|\|\|\|\|\|\|\|\| |
| 11-bp_Stem_G-C_IDX | CACTGACCTCAAGTCTGCACACGAGAAGGCTAG (SEQ ID NO: 215) |
| 11-bp_Stem_G-C_BC01a | CTATCGAGTGCACAT (SEQ ID NO: 216) |
| | \|\|\|\|\|\|\|\|\|\|\|\|\| |
| 11-bp_Stem_G-C_BC01b | ATAGCTCACGTGT (SEQ ID NO: 217) |
| 10-bp_Stem_A-T_UNI | TACACTCTTTCCCTACACGACGCTCTTCCGA (SEQ ID NO: 218) |
| | \|\|\|\|\|\|\|\|\|\| |
| 10-bp_Stem_A-T_IDX | CACTGACCTCAAGTCTGCACACGAGAAGGCTA (SEQ ID NO: 219) |
| 10-bp_Stem_A-T_BC01a | TCTATCGAGTGCACAT (SEQ ID NO: 220) |
| | \|\|\|\|\|\|\|\|\|\|\|\|\|\| |
| 10-bp_Stem_A-T_BC01b | GATAGCTCACGTGT (SEQ ID NO: 221) |
| 9-bp_Stem_A-T_UNI | TACACTCTTTCCCTACACGACGCTCTTCCGA (SEQ ID NO: 222) |
| | \|\|\|\|\|\|\|\|\| |
| 9-bp_Stem_A-T_IDX | CACTGACCTCAAGTCTGCACACGAGAAGGC (SEQ ID NO: 223) |
| 9-bp_Stem_A-T_BC01a | TCTATCGAGTGCACAT (SEQ ID NO: 224) |
| | \|\|\|\|\|\|\|\|\|\|\|\|\|\|\| |
| 9-bp_Stem_A-T_BC01b | TAGATAGCTCACGTGT (SEQ ID NO: 225) |

The ligation reactions were performed as followed:

Amplification and End-Repair of a 300 bp PCR Product from pUC19
1) Q5@ High-Fidelity DNA Polymerase (NEB M0491)—Used standard manufacturer's protocol.
2) NEBNext® Ultra™ II End Repair/dA-Tailing Module (NEB E7546)—Used standard manufacturer's protocol.
3) dA-tailed pcr products were purified using the standard 2×SPRI beads protocol and quantified by QuBit.

Phosphorylation & Annealing of Barcodes
1) 250 pMols of each single stranded barcode was phosphoylated at the 5′-terminus using 0.5 U/μL of T4 PNK (NEB) for 30 min @ 37° C. In 1×T4 DNA ligase buffer (NEB)
2) The barcodes we paired together before denaturing for 2 min @ 80° C. then annealing by cooling to 4° C. and incubating for 10 mins

Adapter Barcode Ligation reaction
1) Barcode and adapter pairs were diluted to 30 μM in 10 mM Tris-HCl and 50 mM NaCl (pH 8.0), before denaturing for 2 min @ 80° C. then annealing by cooling to 4° C. and incubating for 10 mins.
2) Ligation of the 300 bp PCR (50 ng) product from pUC19, with both the barcode (0.25 μM) and adapter (0.25 μM) sequences was achieved in 1×T4 DNA ligase Buffer (NEB), 40 U/μL of T4 DNA ligase (NEB) and incubated for 30 min @ 21° C.
3) The Ligation product was purified using the standard 1×SPRI beads protocol.
4) Indexing PCR was performed with the NEBNext Multiplex Oligos for Illumina kit using the manufacturer's protocol.
5) The indexed PCR product was purified using the standard 1×SPRI beads protocol, before visualizing on an agarose gel.

The agarose gel analysis of the ligation reactions described above are shown in FIG. 5 and FIG. 6. As shown in FIG. 5 and FIG. 6, the partially double-stranded identifier molecules and partially double-stranded adapter molecules can be efficiently ligated to target nucleic acids in the sequencing methods of the present disclosure.

### Example 2—Sequencing Genomic Regions of Interest Using the Sequencing Methods of the Present Disclosure

The following is a non-limiting example of using the compositions and methods of the present disclosure to sequence a plurality of double-stranded target nucleic acid molecules. More specifically, regions of interest were amplified from genomic DNA and analyzed using the compositions and methods of the present disclosure, as well as existing NGS methods, to compare the results of both methods.

Amplification of the Regions of Interest
1) Region of interests were amplified from 5 ng of gDNA (Quantitative Multiplex Reference Standard, Horizon Discovery) using multiplex AmpliSeq PCR primers (0.5 μM), 1×Q5 Reaction Buffer (NEB), 1×Taq Buffer (NEB), 0.2 mM dNTPs (NEB) 1.0 U Q5 Polymerase (NEB) and 1.25 U Taq polymerase (NEB). The PCR mixture was amplified for 2 min at 98° C., then 30 cycles of 30 s at 98° C., 90 s at 60° C. and 30 s at 72° C. and final 5 min at 72° C.
2) The PCR products were purified using the standard 3×SPRI beads protocol.

Adapter Barcode Ligation Reaction and Unique Dual Indexing
1) Ligation of the PCR product (50 ng), with both the barcode (0.25 μM) and adapter (0.25 μM) sequences was achieved in 1×T4 DNA ligase Buffer (NEB), 40 U/μL of T4 DNA ligase (NEB) and incubated for 30 min @ 21° C.
2) The Ligation product was purified using the standard 1×SPRI beads protocol.
3) Indexing PCR was performed with the NEBNext Multiplex Oligos for Illumina kit using the manufacturer's protocol.
4) The indexed PCR product was purified using the standard 1×SPRI beads protocol, before visualizing on an agarose gel and sequenced

Generation of Duplex Consensus Sequence BAM Files, Starting with Paired-End Fastq Files
1) Fastq files generated from Next-generation Sequences were processed according to the workflow shown in FIG. 4, including the CorrectUmis and GroupBySeq steps:
   a) CorrectUmis—The partially double stranded identifier molecules and adapter molecules used to generate UMIs (i.e. the specific combination of the two species of partially double-stranded identifier molecules that are ligated to a single transcript), can further be used for error correction (once assembled libraries have been sequenced), due to the fact that the UMIs created are discrete with a defined distance between each UMI (see FIG. 7). Error-correction is not normally applied to UMIs generated from a pre-pooled, degenerate method due to there being no control over the distance between UMIs.
   b) GroupBySeq—Using EGFR4 as an example and focusing on the G>A variant present at an allelic frequency of 24.5%, single stranded consensus sequences (SSCS) were generated using CallMolecularConsensus (fgbios tools) and visualized via IGV (Broad Institute). Overall, base calling at the known G>A variant position, was of a lower quality for SSCS generated without the GroupBySeq step (top), than compared to SSCS generated with the GroupBySeq step.
      Without wishing to be bound by theory, for existing pre-pooled, degenerate UMIs, the number of UMIs available should be larger than the number of molecules present within the initial sample. This ensures each molecule gets a unique UMI. This approach leads to a large majority of UMIs containing only a single read. With a minimum requirement of at least two reads per UMI, to generate a consensus sequence, the UMIs containing only a single read are discarded. The inability to produce a consensus read for a large majority of the available UMIs, means that very high sequencing depths are required for each region of interest. These limitations are overcome by the methods of the present disclosure by:—
         1) giving the user the ability to modulate the number of discrete UMIs used for the initial sample;
         2) using the sequence from the region of interest as a UMI itself Aligned reads can be grouped together based on their absolute alignment to the reference using GroupBySeq, within GroupBySeq reads are grouped based on their similarity/difference from the reference. The GroupBySeq reads can then further be sub-divided by their UMIs. Because the number of initial UMIs can be modulated, the number of reads per UMI can be modulated to have on average at least two reads per UMI (once the GropBySeq step has been performed). Optimization of the number of reads per UMI, allows the majority of reads (and therefore UMIs) to produce usable consensus reads, therefore reducing the coverage required per region of interest.

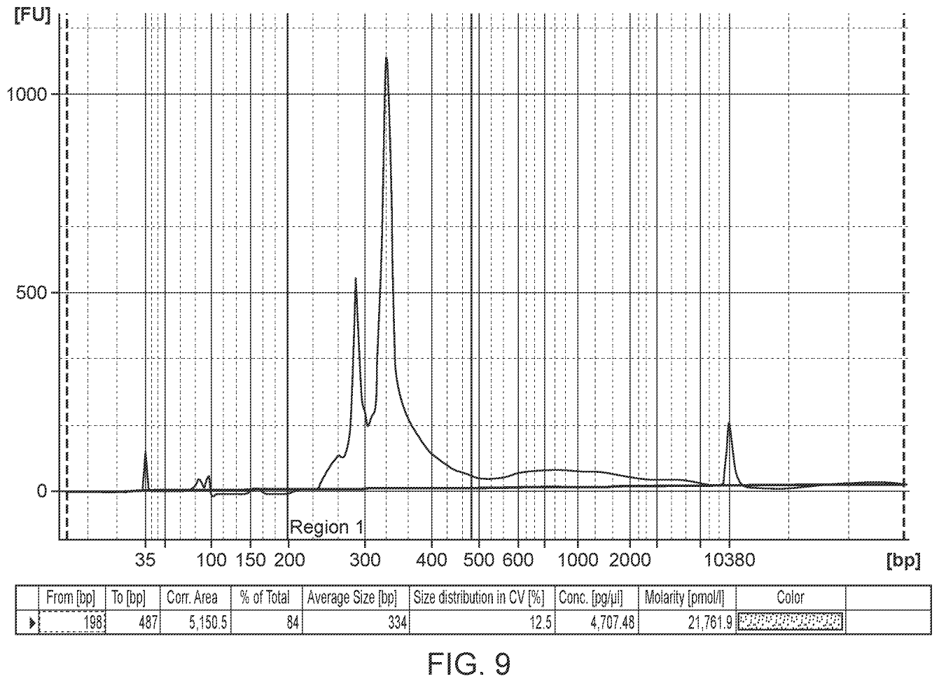

The sequencing libraries created using the sequencing methods of the present disclosure were analyzed using a Bioanalyzer system. The results of the analysis are shown in FIG. 9. As shown in FIG. 9, minimal adapter dimers were observed in the 100-150 bp region.

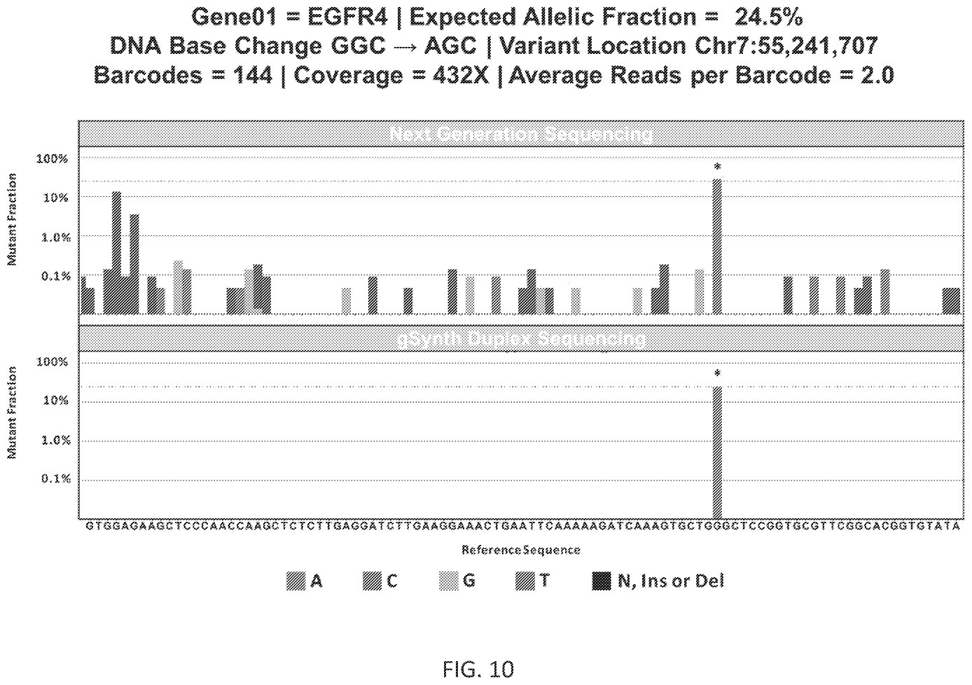

The analysis of the sequencing results for specific mutations in 11 genes are shown in FIGS. 10-20, including the results using existing NGS methods (top panel) and the results using the sequencing methods of the present disclosure (denoted gSynth Duplex Sequencing in FIGS. 10-20). FIGS. 10-20 also show the expected allelic fraction of the mutation that is being analyzed, and the number of different UMIs (barcodes) that are possible based on the number of species of partially double-stranded identifier molecules that were used in the sequence (e.g. 12 species yield 144 possible barcodes, 24 species yield 576 possible barcodes, 48 species yield 2,304 possible barcodes, etc.).

FIG. 10 shows the sequencing results for the EGFR4 gene and the measured mutant frequencies for a DNA base change of GGC→AGC.

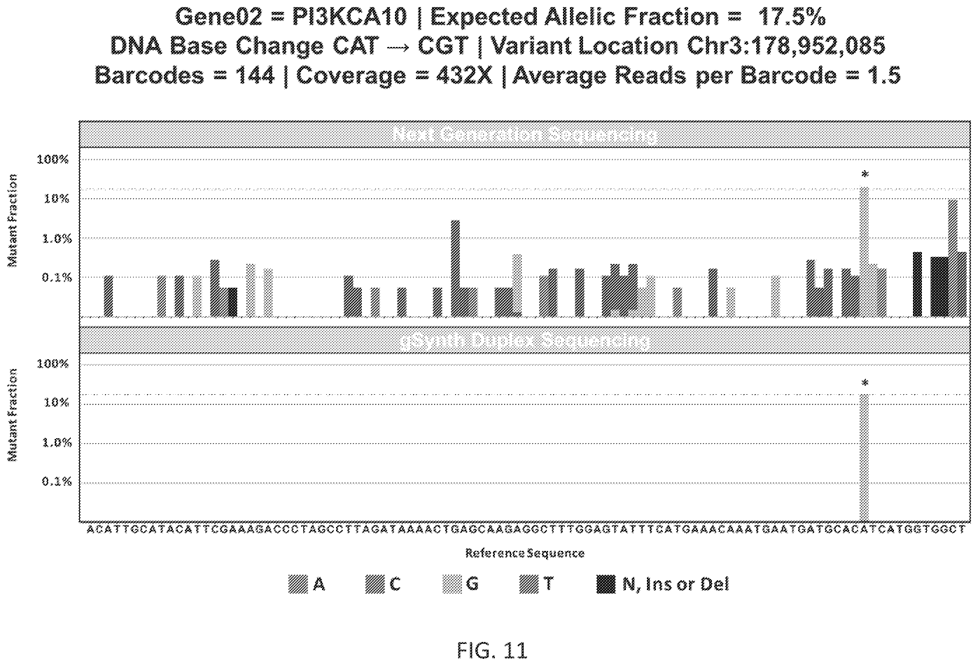

FIG. 11 shows the sequencing results for the PI3KCA10 gene and the measured mutant frequencies for a DNA base change of CAT→CGT.

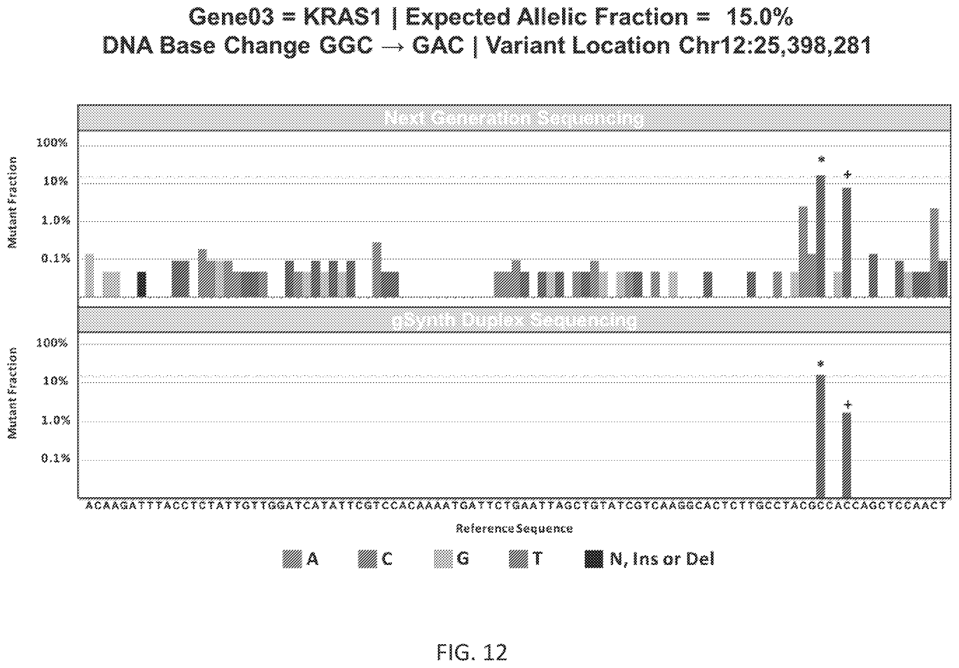

FIG. 12 shows the sequencing results for the KRAS1 gene and the measured mutant frequencies for a DNA base change of GGC→GAC.

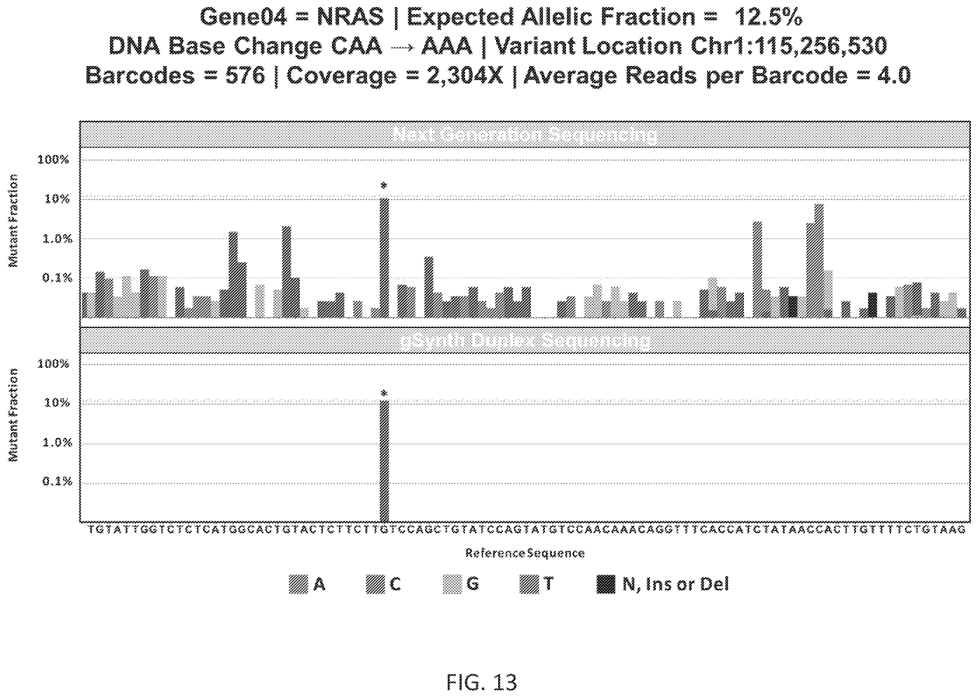

FIG. 13 shows the sequencing results for the NRAS gene and the measured mutant frequencies for a DNA base change of CAA→AAA.

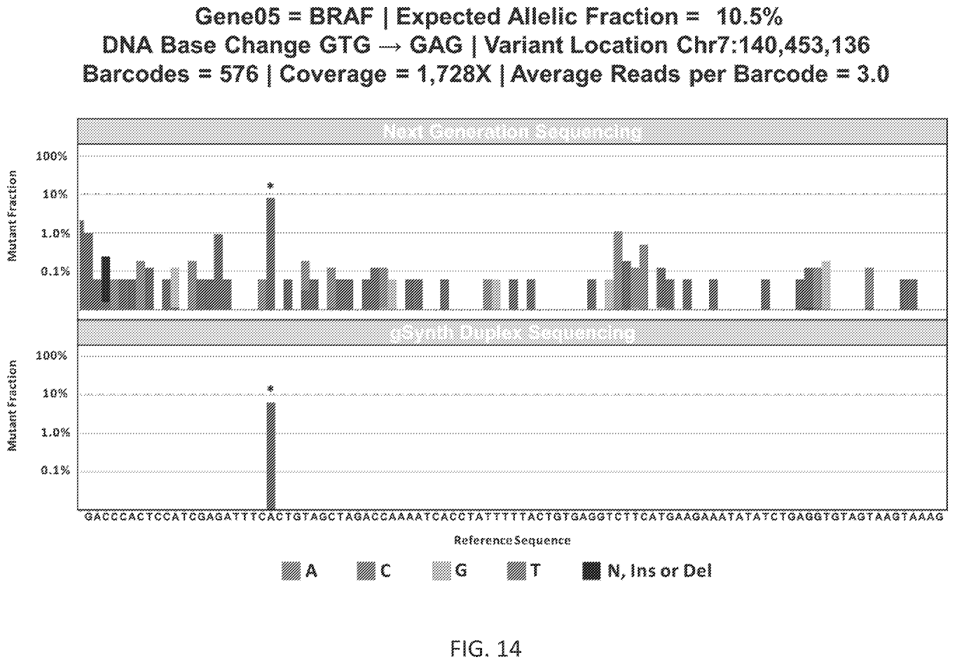

FIG. 14 shows the sequencing results for the BRAF gene and the measured mutant frequencies for a DNA base change of CTG→CAG.

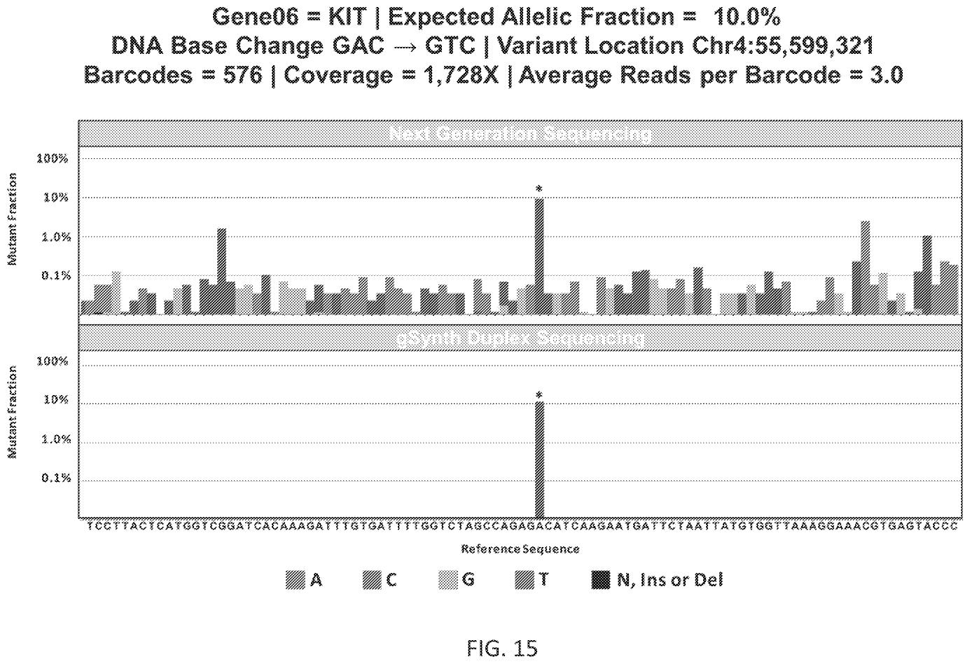

FIG. 15 shows the sequencing results for the KIT gene and the measured mutant frequencies for a DNA base change of GAC→GTC.

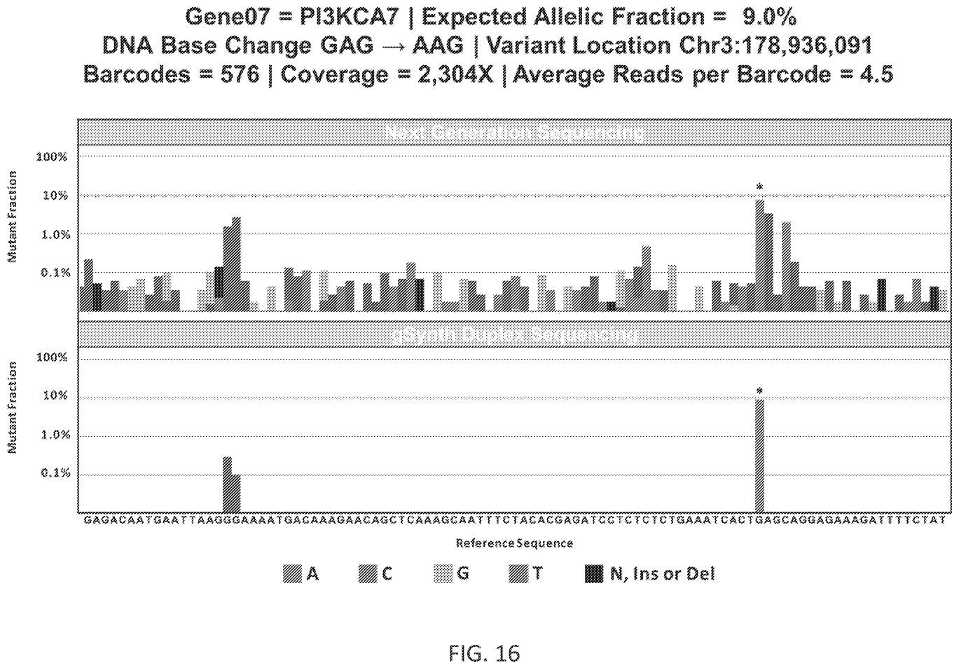

FIG. 16 shows the sequencing results for the PI3KCA7 gene and the measured mutant frequencies for a DNA base change of GAG→AAG.

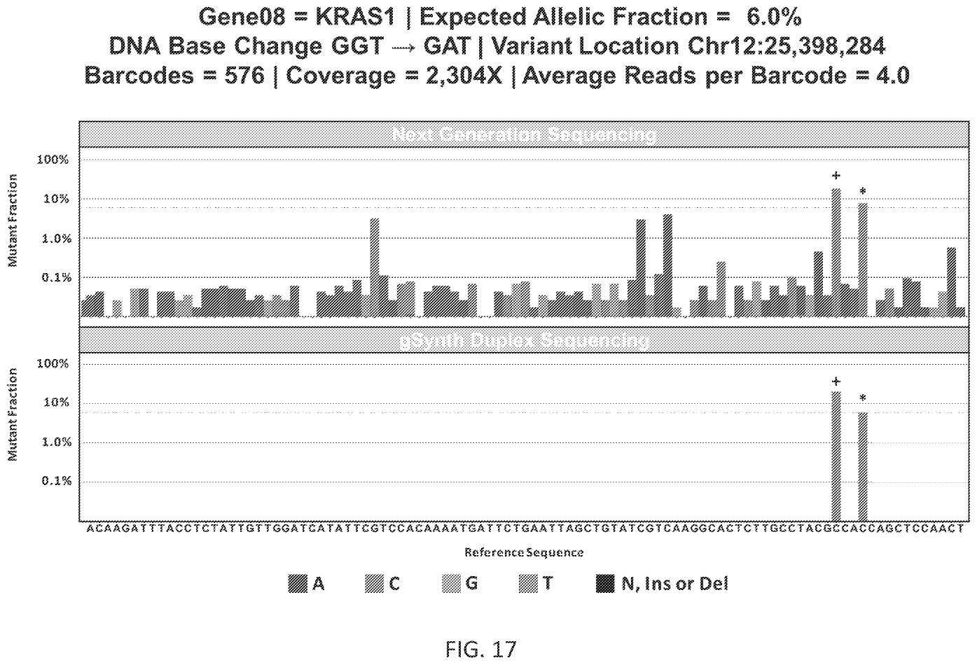

FIG. 17 shows the sequencing results for the KRAS1 gene and the measured mutant frequencies for a DNA base change of GGT→GAT.

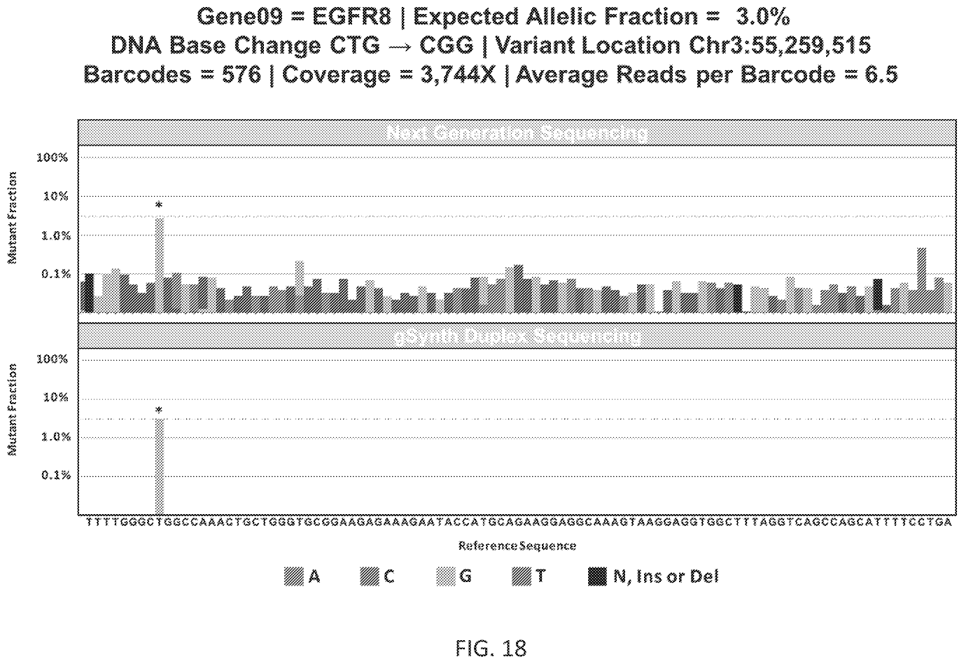

FIG. 18 shows the sequencing results for the EGFR8 gene and the measured mutant frequencies for a DNA base change of CTG→CGG.

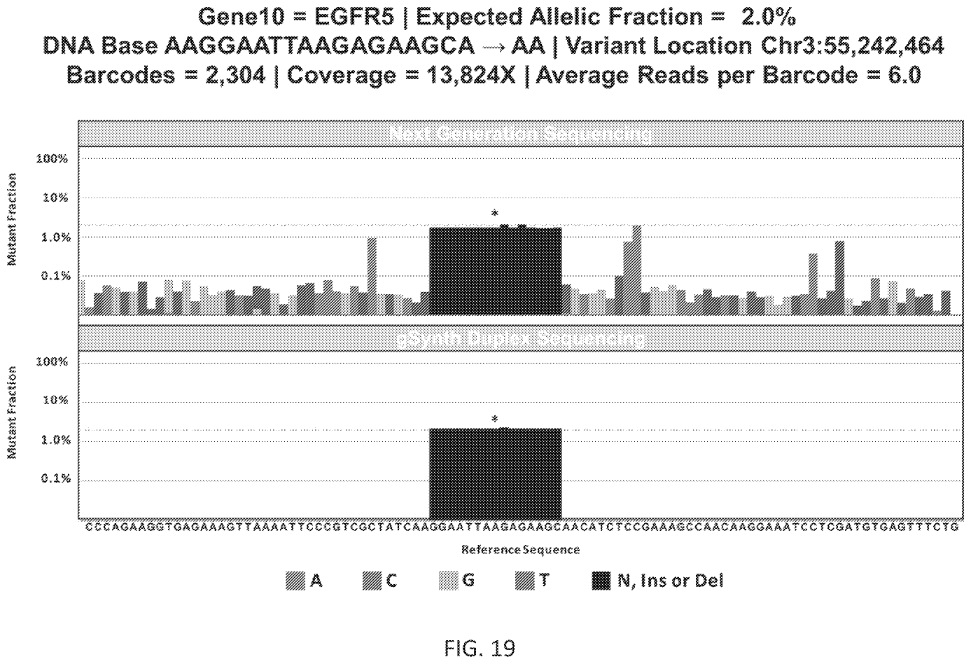

FIG. 19 shows the sequencing results for the EGFR5 gene and the measured mutant frequencies for a DNA base change of AAGGAATTAAGAGAAGCA→AA.

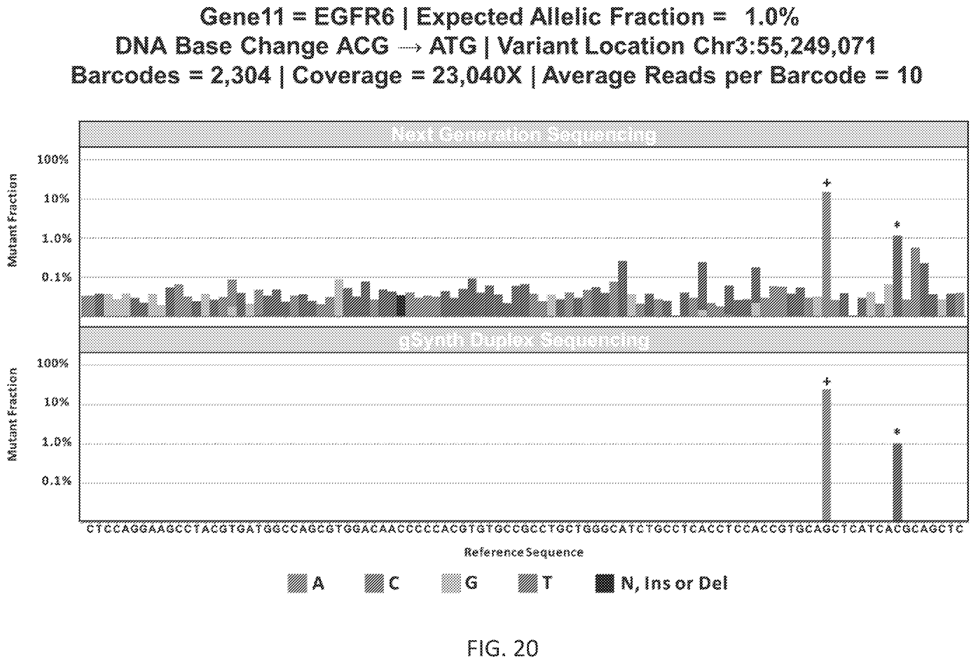

FIG. 20 shows the sequencing results for the EGFR6 gene and the measured mutant frequencies for a DNA base change of ACG→ATG.

As shown in FIGS. 10-20, the sequencing results obtained by the methods of the present disclosure, and more specifically the mutation frequency measured using the sequencing methods of the present disclosure was more accurate as compared to the results obtained using existing NGS methods. Moreover, the sequencing results obtained by the methods of the present disclosure exhibited less noise as compared to the sequencing results obtained by existing NGS methods. Accordingly, the results presented in this example demonstrate that the sequencing compositions and methods of present disclosure provide superior sequencing results, including mutation frequency measurements, as compared to existing NGS methods.

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A plurality of partially double-stranded identifier molecules,
wherein the partially double-stranded identifier molecules comprise:
a double-stranded region comprising an identifier sequence; and
a first overhang;
wherein the plurality comprises at least about 12 species of the partially double-stranded adapter molecules, wherein each species of partially double-stranded adapter molecules comprises an identifier sequence that is different from the identifier sequence of any other species of partially double-stranded adapter molecules in the plurality,
wherein the identifier sequence of one species of partially double-stranded identifier molecules will have a hamming distance of at least about two to any other identifier sequence of any other species of partially double-stranded identifier molecules in the plurality.

2. The plurality of partially double-stranded identifier molecules of claim 1, wherein the partially double-stranded identifier molecules further comprise a second overhang.

3. The plurality of partially double-stranded identifier molecules of any of the preceding claims, wherein the first and/or second overhangs are
a) 5′ overhangs; or
b) 3′ overhangs.

4. The plurality of partially double-stranded identifier molecules of any of the preceding claims, wherein:
a) the identifier sequence spans the entire double-stranded region; or
b) the identifier sequence spans a portion of the double-stranded region.

5. The plurality of partially double-stranded identifier molecules of any of the preceding claims, wherein the identifier sequence is:
a) about 9 nucleotides in length;
b) about 10 nucleotides in length:
c) about 11 nucleotides in length;
d) about 12 nucleotides in length;
e) about 19 nucleotides in length;
f) about 20 nucleotides in length;
g) about 21 nucleotides in length; or
h) about 22 nucleotides in length.

6. The plurality of partially double-stranded identifier molecules of any of the preceding claims, wherein the first overhang and/or the second overhang is about 1 nucleotide in length, preferably wherein the first overhang and/or the second overhang is:
a) an adenine or a thymine; or
b) a guanosine or a cytosine.

7. The plurality of partially double-stranded identifier molecules of any of the preceding claims, wherein the first overhang and/or the second overhang is:
a) about 2 nucleotides in length;
b) about 3 nucleotides in length;
c) about 4 nucleotides in length; or
d) about 5 nucleotides in length.

8. The plurality of partially double-stranded identifier molecules of any of the preceding claims, wherein the partially double-stranded identifier molecules comprise DNA.

9. The plurality of partially double-stranded identifier molecules of any of the preceding claims, wherein the plurality comprises:
a) at least about 24 species of the partially double-stranded identifier molecules;
b) at least about 48 species of the partially double-stranded identifier molecules; or
c) at least about 96 species of the partially double-stranded identifier molecules.

10. A plurality of partially double-stranded adapter molecules,
wherein the partially double-stranded adapter molecules comprise:
a double-stranded region;
an overhang;
a single-stranded 5′ arm; and
a single-stranded 3′ arm;
wherein the single-stranded 5′ arm comprises at least one amplification primer binding site and the single-stranded 3′ arm comprises at least one amplification primer binding site.

11. The plurality of partially double-stranded adapter molecules of claim 10, wherein the double-stranded region comprises an identifier sequence,
wherein the plurality comprises at least about 12 species of the partially double-stranded adapter molecules, wherein each species of partially double-stranded adapter molecules comprises an identifier sequence that is different from the identifier sequence of any other species of partially double-stranded adapter molecules in the plurality.

12. The plurality of partially double-stranded adapter molecules of any one of claims 10-11, wherein the overhang is:
a) a 5′ overhang; or
b) a 3′ overhang.

13. The plurality of partially double-stranded adapter molecules of any one of claims 10-12, wherein the overhang is about 1 nucleotide in length, preferably wherein the overhang is:
a) an adenine or a thymine; or
b) a guanosine or cytosine.

14. The plurality of partially double-stranded adapter molecules of any one of claims 10-12, wherein the overhang is:
a) about 2 nucleotides in length;
b) about 3 nucleotides in length;
c) about 4 nucleotides in length;
d) about 5 nucleotides in length.

15. The plurality of partially double-stranded adapter molecules of any one of claims 11-14, wherein the identifier sequence is
a) about 9 nucleotides in length;
b) about 10 nucleotides in length:
c) about 11 nucleotides in length;
d) about 12 nucleotides in length;
e) about 19 nucleotides in length;
f) about 20 nucleotides in length;
g) about 21 nucleotides in length; or
h) about 22 nucleotides in length.

16. The plurality of partially double-stranded adapter molecules of any one of claims 10-15, wherein the partially double-stranded adapter molecules comprise DNA.

17. A method of sequencing a plurality of double-stranded target nucleic acids comprising:
a) contacting the plurality of double-stranded target nucleic acids with the plurality of partially double-stranded identifier molecules of any one of claims 1-9 and at least one ligase such that a partially double-stranded identifier molecule is ligated to each end of the double-stranded target nucleic acids in the plurality of double-stranded target nucleic acids;
b) contacting the products of step (a) with the plurality of partially double-stranded adapter molecules of any one of claims 10-16 and at least one ligase such that a partially double-stranded adapter molecule is ligated to each end of the products of step (a); and
c) sequencing the products of step (b).

18. The method of claim 17, wherein the ligation products in step (a) comprise:
a) at least 10% of the combinations of two species of partially double-stranded identifier molecules;
b) at least 20% of the combinations of two species of partially double-stranded identifier molecules;
c) at least 30% of the combinations of two species of partially double-stranded identifier molecules;
d) at least 40% of the combinations of two species of partially double-stranded identifier molecules;
e) at least 50% of the combinations of two species of partially double-stranded identifier molecules;
f) at least 60% of the combinations of two species of partially double-stranded identifier molecules;
g) at least 70% of the combinations of two species of partially double-stranded identifier molecules;
h) at least 80% of the combinations of two species of partially double-stranded identifier molecules;
i) at least 90% of the combinations of two species of partially double-stranded identifier molecules; or
j) each of the combinations of two species of partially double-stranded identifier molecules.

19. The method of claim 17 or claim 18, the method further comprising after step (b) and prior to step (c), constructing a sequencing library using the products of step (b).

20. The method of any one of claims 17-19, wherein step (a) and step (b):
a) are performed sequentially; or
b) are performed concurrently.

21. The method of any one of claims 17-20, the method further comprising after step (b) and prior to step (c), amplifying the products of step (b), preferably wherein amplifying the products of step (b) comprises contacting the products of step (b) with amplification primers that bind to amplification primer binding sites in the partially double-stranded adapter molecules and at least one polymerase.

22. The method of any one of claims 17-21, wherein the method further comprising determining the abundance and/or the identity of specific transcripts in the plurality of double-stranded target nucleic acids using the sequencing data generated in step (c), preferably wherein determining the abundance and/or the identity of specific transcripts in the plurality of double-stranded target nucleic acids using the sequencing data generated in step (c) comprises correcting for errors using the identifier sequences of the ligated partially double-stranded identifier molecules, preferably wherein the errors comprise amplification errors, sample preparation errors, sequencing errors or any combination thereof, and/or determining the abundance and/or the identity of specific transcripts in the plurality of double-stranded target nucleic acids using the sequencing data generated in step (c) comprises creating consensus sequences using identifier sequences of the ligated partially double-stranded identifier molecules.

23. The method of claim 22, wherein determining the abundance and/or identity of specific transcripts in the plurality of double-stranded target nucleic acids using the sequencing data generated in step (c) comprises
a) grouping the sequencing reads obtained in step (c) by the ligated identifier sequences in the sequencing reads;
b) grouping the sequencing reads obtained in step (c) by the specific genomic sequence that the sequencing reads most likely correspond to; or
c) any combination thereof.

24. The method of claim 22 or claim 23, wherein determining the abundance and/or identify of specific transcripts in the plurality of double-stranded target nucleic acids can comprise determining the frequency of one or more mutations in a specific transcript in the plurality of double-stranded target nucleic acid, preferably wherein the one or more mutations comprise one or more insertions, one or more deletion-insertions, one or more duplications, one or more inversions, one or more repeat expansions or any combination thereof.

25. A kit comprising the plurality of partially double-stranded identifier molecules of any one of claims 1-9.

26. The kit of claim 25, wherein the kit further comprises the plurality of partially double-stranded adapter molecules of any one of claims 10-16.
